(19) United States
(12) Patent Application Publication
(10) Pub. No.: US 2020/0325232 A1
(43) Pub. Date: Oct. 15, 2020

(54) MULTISPECIFIC ANTIGEN BINDING PROTEINS

(71) Applicant: INNATE PHARMA, MARSEILLE (FR)

(72) Inventor: LAURENT GAUTHIER, MARSEILLE (FR)

(21) Appl. No.: 16/765,486

(22) PCT Filed: Nov. 19, 2018
(86) PCT No.: PCT/EP2018/081814

§ 371 Date: May 20, 2020

Related U.S. Application Data
(60) Provisional application No. 62/588,952, filed on Nov. 21, 2017.

(51) Int. Cl. C07K 16/28; A61P 35/00; G01N 33/68

(52) U.S. Cl. CPC C07K 16/2851; C07K 16/283; A61P 35/00; G01N 33/6854; C07K 2317/622; C07K 2317/522; C07K 2317/526; C07K 2317/64; C07K 2317/732; C07K 2317/31

(57) ABSTRACT

Multimeric multispecific proteins that bind multiple target antigens are provided. The proteins have particular advantages when configured to bind a NK or T cell activating receptor and one or two cancer associated antigens, and can be used in the treatment of disease, notably cancer.

### CROSS-REFERENCE TO RELATED APPLICATIONS

This application claims the benefit of U.S. Provisional Application No. 62/588,952 filed Nov. 21, 2017, which is incorporated herein by reference in its entirety; including any drawings and sequence listings.

### REFERENCE TO THE SEQUENCE LISTING

The present application is being filed along with a Sequence Listing in electronic format. The Sequence Listing is provided as a file entitled “BISP6_ST25 txt”, created 19 Nov. 2018, which is 61 KB in size. The information in the electronic format of the Sequence Listing is incorporated herein by reference in its entirety.

### FIELD OF THE INVENTION

Multispecific proteins that bind and can be used to specifically redirect effector cells to lyse a target cell of interest are provided. The proteins formats have utility in the treatment of disease.

### BACKGROUND

Bispecific antibodies binding two different epitopes and offer opportunities for increasing specificity, broadening potency, and utilizing novel mechanisms of action that cannot be achieved with a traditional monoclonal antibody. A variety of formats for bispecific antibodies that bind to two targets simultaneously have been reported. Cross-linking two different receptors using a bispecific antibody to inhibit a signaling pathway has shown utility in a number of applications (see, e.g., Jackman, et al., (2010) J. Biol. Chem. 285:20850-20859). Bispecific antibodies have also been used to neutralize two different receptors. In other approaches, bispecific antibodies have been used to recruit immune effector cells, where T-cell activation is achieved in proximity to tumor cells by the bispecific antibody which binds receptors simultaneously on the two different cell types (see Baeuerle, P. A., et al, (2009) Cancer Res 69(12):4941-4). Most such approaches involve bispecific antibodies that link the CD3 complex on T cells to a tumor-associated antigen. The most well-studied bispecific antibody formats are “BiTe” antibodies and “DART” antibodies which do not comprise Fc domains. However these antibodies are known to be difficult to produce, require lengthy cell development, have low productions yields and/or cannot be produced (based on published literature) as a homogenous protein composition. Notably, in order to fully activate a T-cell, the T-cell and a cluster of BiTEs must interact on the surface of a target cell. Due to the difficulties of finding antibody variable regions which are functional in the BITE format, to date only a single immune cell receptor (CD3) has been targeted, in the CD19×CD3 specific antibody blinatumamab. Bispecific antibodies developed to date also include those which link the CD3 complex on T cells to a tumor-associated antigen. In another example, a bispecific antibody having one arm which bound FcγRIII and another which bound to the HER2 receptor was developed for therapy of ovarian and breast tumors that overexpress the HER2 antigen.

However, despite the existence of a variety of formats for bispecific antibodies, there is therefore a need in the art for proteins with new and well-defined mechanisms of action that can bind two or more biological targets, and that have attractive properties for industrial development.

### SUMMARY OF THE INVENTION

The present invention arises from the discovery of functional protein structures that permits a wide range of antibody variable regions or other antigen binding domains to be integrated therein, and moreover does not require mutated or non-natural protein domains (e.g. wild-type CH1, CK and/or Fc domains can be used) and make minimal use of non-immunoglobulin-derived linker sequences. The multimeric protein constructions are designed to drive the preferential association of polypeptide chains into the desired configuration, while being adapted to standard recombinant production techniques and without the need for development of product-specific folding or purification technique. While the new protein formats can be used to bind any desired antigens by incorporation the desired variable regions or other suitable binding domains, advantageous examples are provided where multispecific proteins can bind to one or more antigens (e.g. two different antigens co-expressed on the surface of target cells (e.g. cells to be eliminated or depleted, cancer promoting cells, tumor cells, cells in tumor tissue or tumor adjacent tissue), and a receptor (e.g. an activating receptor) on an immune cell (e.g., a leucocyte, a lymphocyte, an effector cell, a NK cell, a T cell, etc.). In one embodiment, the activating receptor is a human cytotoxicity receptor selected from the group consisting of: NKp46, NKp30, NKp44, CD137, CD3, CD8 and NKG2D. The protein can further bind, via its dimeric Fc domain, to FcRn, and optionally further to CD16A and/or other Fcγ receptors.

In some particularly advantageous examples, the proteins possess two antigen binding domains (ABDs) that each bind to a target antigen (e.g. cancer antigen), a dimeric Fc domain that binds human FcRn (and optionally further the activating receptor CD16A), and an ABD that binds an effector cell activating receptor other than CD16A (or more generally, other than a Fcγ receptor). When the ABDs that bind a cellular target antigen (e.g. antigen expressed at the surface of a cancer cell) are positioned in cis with respect to the Fc domain (positioned on the same side or terminus of the dimeric Fc domain) and the ABD that binds the effector cell activating receptor is placed in trans with respect to the ABD that bind the target antigen (positioned on the opposite terminus of the dimeric Fc domain), the protein has increased ability to cause immune cell mediated cytotoxicity toward the cell expressing the target antigen. In such a configuration each ABD can be fused, via a CH1 or CK domain, to the N terminus of an Fc domain, and rather than a decrease in avidity due to steric hindrance, the avidity of the protein for the target cell was found to be increased.

Further advantageously, the instant proteins can be configured to have, in addition to an ABD that binds an effector cell activating receptor other than CD16A (e.g., NKp46, NKp30, NKG2D, CD137, etc.), two antigen binding domains (ABDs), wherein each ABD binds to a different target antigen (e.g. cancer antigen) expressed by a cell (target cell; a cancer-promoting cell, cancer cell, a cell in tumor tissue or tumor adjacent tissue, or any other desired cell population). The ability of the multispecific protein to bind two different target antigens expressed (co-expressed) at the surface of a cell can increase in number of receptors available at the cell surface for binding by the protein, and in avidity for binding to the cell. Furthermore, the two different target antigens can be selected to be cancer antigens that are expressed (co-expressed) at the surface of a target cell (e.g. a cancer cell, a cancer-promoting cell, a cell present in tumor tissue or tumor adjacent tissue) but are not both expressed at the surface of healthy (non-cancer) cells. Such a selection of binding specificity permits the incorporation of an ABD that binds a target antigen which is expressed on healthy tissues or cells (in addition to expression on disease cells such as tumor or cancer promoting cells), so long as both antigens are not co-expressed on the healthy cells.

In one embodiment, provided is a hetero-multimeric multispecific protein comprising: a first antigen binding domain (ABD₁) that specifically binds to a first antigen of interest, a second antigen binding domain (ABD₂) that specifically binds a second antigen of interest, and a third antigen binding domain (ABD₃) that specifically binds a third antigen of interest, and at least a portion of a human Fc domain, wherein the Fc domain is interposed between ABD₁ and the ABD₂ and ABD₃ pair (i.e. ABD₁ is placed C-terminal to the Fc domain and ABD₂ and ABD₂ are placed N-terminal of the Fc domain, or vice-versa. In one example, the first antigen is an antigen expressed by an immune effector cell (e.g. an NK cell and/or a T cell), the second antigen is an antigen expressed by a target cell to be eliminated, and the third antigen is an antigen expressed by a target cell to be eliminated (either the same of different from the second antigen, or a different epitope on the same protein as the second antigen). Consequently, the multispecific protein can bind a target cell to be eliminated in bivalent manner, and bind the antigen expressed by an immune effector cell in monovalent manner. Such a multispecific protein may permit advantageous targeting of an antigen expressed by target cell by triggering an activating receptor on an effector cell in monovalent manner, thereby preventing or reducing agonist activity at the receptor on effector cells in the absence of target cells.

In one aspect, provided is a multispecific antigen binding protein comprising a first antigen binding domain (“ABD”) that binds to a human NKp46 protein, a second ABD that binds to a first pre-determined antigen of interest, and a third ABD that binds to a second, different, pre-determined antigen of interest, and a CD16A binding polypeptide, optionally an Fc polypeptide or portion thereof capable of binding human CD16A which Fc polypeptide optionally is modified to enhance CD16A binding, wherein the first antigen binding domain is positioned C-terminal to the CD16A binding polypeptide and the second and third ABD are both positioned N-terminal to the CD16A binding polypeptide, wherein the multispecific protein is capable of directing an NKp46-expressing NK cell to lyse a target cell co-expressing the first and second antigen of interest, wherein said lysis of the target cell is mediated by NKp46-signaling.

In one aspect, provided is a multimeric (e.g. heterodimeric or heterotimeric) protein that possess two antigen binding domains (ABDs), where each ABD binds to a different target antigen co-expressed at the surface of a target cell to be eliminated (e.g. a pro-tumoral cell, a cancer cell, a cell present in tumor tissue or tumor adjacent tissue), optionally wherein one or both of the target antigens are known to also be expressed by healthy cells, a dimeric Fc domain that binds human FcRn (and optionally further the activating receptor CD16A), and an ABD that binds a human cytotoxicity receptor selected from the group consisting of: NKp46, NKp30, NKp44, CD137, CD3, CD8 and NKG2D. In certain embodiments, multimeric polypeptides can be constructed such that they are composed of three different polypeptide chains in which one chain dimerizes with a central chain based on CH1-CK heterodimerization and CH3-CH3 dimerization, and another chain dimerizes with a central chain based on CH1-CK hereodimerization and VH-VK association.

In one aspect of any embodiment herein, the multispecific protein binds to an activating receptor on an immune effector cell (e.g. NKp46, NKp30, NKp44, CD137, CD3, CD8 or NKG2D) in monovalent manner. In one embodiment, the multispecific protein is capable of mediating agonist activity (e.g. triggering signaling) of the activating receptor (e.g. NKp46, NKp30, NKp44, CD137, CD3, CD8 or NKG2D) in an immune effector cell expressing the activating receptor in the presence of a target cell (e.g. a cell to be eliminated that expresses an antigen bound by the multispecific protein). Optionally, the multispecific protein is capable of mediating agonist activity of the activating receptor(s) in an immune effector cell expressing the activating receptor and a target cell, yet does not substantially induce or mediate agonist activity of the activating receptor(s) (e.g., the respective NKp46, NKp30, NKp44, CD137, CD3, CD8 or NKG2D) in an immune effector cell expressing the activating receptor in the absence of a target cell. Agonist activity can be assessed by any suitable method, e.g. stimulation of activating-receptor dependent target cell lysis by an immune effector cell, activation and/or cytotoxicity markers on an immune cell, assessment of signaling or signaling pathways by the activating receptor, etc.

In one embodiment, provided is a multispecific protein that binds a first, second and third antigen of interest, comprising a first, second and third polypeptide chain, comprising:

a first (central) chain comprising, from N- to C-terminus, an antigen binding domain that binds the second antigen of interest, a first CH1 or Cκ domain, an Fc region, and a variable domain fused to a second CH1 or Cκ domain (forming a V-(CH1/Cκ) unit);

a second chain comprising, from N- to C-terminus, a variable domain fused to a CH1 or Cκ domain, wherein the variable domain and CH1 or Cκ domain are complementary to the respective variable domain and second CH1 or Cκ domain of the first chain (the V-(CH1/Cκ) unit), such that the second chain binds to the first chain by CH1-Cκ dimerization and VH-VK association, wherein the VH and VK together form a first antigen binding domain that binds a the first antigen of interest; and a third chain comprising, from N- to C-terminus, an antigen binding domain that binds the third antigen of interest, a CH1 or Cκ domain and a Fc region, wherein said CH1 or Cκ domain is complementary to the first CH1 or Cκ domain of the first chain, such that the third chain binds to the first chain by CH1-Cκ dimerization and CH3-CH3 dimerization.

In one embodiment, provided is a heterotrimeric multispecific protein comprising a dimeric Fc domain, having the domain arrangement:

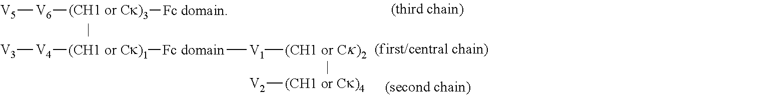

wherein one of (CH1 or Cκ)₁ and (CH1 or Cκ)₃ is a CH1 domain and the other is a Cκ domain, wherein one of (CH1 or Cκ)₂ and (CH1 or Cκ)₄ is a CH1 domain and the other is a Cκ domain, wherein one V₁ and V₂ is a light chain variable domain and the other is a heavy chain variable domain, wherein the V₁ and V₂ pair associate with one another to form an ABD₁ that binds a first antigen of interest, wherein one V₃ and V₄ is a light chain variable domain and the other is a heavy chain variable domain, wherein the V₃ and V₄ pair associate with one another to form an ABD₂ that binds a second antigen of interest, and wherein one V₅ and V₆ is a light chain variable domain and the other is a heavy chain variable domain, wherein the V₅ and V₆ pair associate with one another to form an ABD₃ that binds a third antigen of interest. Optionally, each (CH1 or Cκ)₁ and (CH1 or Cκ)₃ is fused to the Fc domain via an immunoglobulin hinge amino acid sequence. Optionally, ABD₁ binds specifically to an activating receptor expressed at the surface of an immune cell, ABDₛ binds specifically to a first cancer antigen, and ABD₂ binds specifically to a second cancer antigen, wherein the first and second cancer antigen are different from one another, optionally wherein the first and second cancer antigen are co-expressed by a malignant cell or tumor-promoting cell, optionally further wherein one of the first and second cancer antigen are known to be expressed at the surface of healthy cells.

In another embodiment, a multispecific protein that binds a first, second and third antigen of interest is a heterodimer protein comprising the domain arrangement:

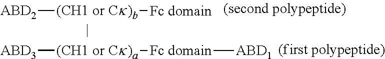

In these structures, the Fc domains of the first and second chains associate via CH3-CH3 dimerization, and (CH1 or Cκ)_{b} on the second chain and the (CH1 or Cκ)ₐ on the first chain undergo CH1-Cκ dimerization, wherein ABD₁ that binds an activating receptor on an immune cell (e.g. NKp46) and ABD₂ and ABD₃ are each self-contained antigen binding domains that can bind an antigen of interest (e.g. a cancer antigen) without association with a complementary domain on a different polypeptide chain, wherein each (CH1 or Cκ)_{b} and (CH1 or Cκ)ₐ is fused to the Fc domain via an immunoglobulin hinge amino acid sequence. Optionally, ABD₁ binds specifically to an activating receptor expressed at the surface of an immune cell, ABDₛ binds specifically to a first cancer antigen, and ABD₂ binds specifically to a second cancer antigen, wherein the first and second cancer antigen are different from one another, optionally wherein the first and second cancer antigen are co-expressed by a malignant cell or tumor-promoting cell, optionally further wherein one of the first and second cancer antigen are known to be expressed at the surface of healthy cells.

Optionally in any embodiment, each antigen binding domain comprises the hypervariable regions, optionally the heavy and light chain CDRs, of an antibody. Optionally in any embodiment, a variable domain comprises framework residues from a human framework region, e.g., a variable domain comprises 1, 2 or 3 CDRs of human or non-human origin and framework residues of human origin.

In one embodiment of any of the polypeptides herein, the multispecific protein is capable of directing effector cells (e.g. a T cell, an NK cell) expressing one of the antigens of interest to lyse a target cell expressing the other of the antigens of interest (e.g. a cancer cell, a virally infected cell, a bacterial cell, a pro-inflammatory cell, etc.).

In one embodiment of any of the polypeptides herein, the multispecific protein comprises a dimeric Fc domain capable of binding to human CD16, and the protein is capable of directing effector cells (e.g. a T cell, an NK cell) that express human CD16 to lyse a target cell expressing one or more of the antigens of interest (e.g. a cancer cell). In one embodiment, the multispecific protein causes lysis of the target cell at least in part by CD16-mediated antibody-dependent cell-mediated cytotoxicity (“ADCC”). In one embodiment, the multispecific protein causes lysis of the target cell by a combination of (a) enhancing or inducing signaling of an activating receptor on immune cells bound by an ABD of the multispecific protein, and (b) CD16-mediated antibody-dependent cell-mediated cytotoxicity (“ADCC”).

In one aspect the invention provides an isolated multispecific heterotrimeric protein comprising a first polypeptide chain comprising an amino acid sequence which is at least 50%, 60%, 70%, 80%, 85%, 90%, 95%, 98 or 99% identical to the sequence of a first polypeptide chain of a F18, F19 or F20 protein disclosed herein (e.g. a polypeptide of SEQ ID NO: 19, 22 or 25, respectively); a second polypeptide chain comprising an amino acid sequence which is at least 50%, 60%, 70%, 80%, 85%, 90%, 95%, 98 or 99% identical to the sequence of a second polypeptide chain of the respective F18, F19 or F20 protein disclosed herein (e.g. a polypeptide of SEQ ID NO: 20, 23 or 26, respectively); and optionally a third polypeptide chain comprising an amino acid sequence which is at least 50%, 60%, 70%, 80%, 85%, 90%, 95%, 98 or 99% identical to the sequence of a third polypeptide chain of a F18, F19 or F20 protein disclosed herein (e.g. a polypeptide of SEQ ID NO: 18, 21 or 24, respectively). In one embodiment, variable regions are excluded from the sequences that are considered for computing sequence identity. In one aspect, the protein comprises a dimeric Fc domain capable of being bound by a human CD16 polypeptide, e.g. a dimeric Fc domain comprising N-linked glycosylation at residue N297 (Kabat EU numbering). Optionally any or all of the variable regions or CDRs of the first, second and/or third chains are substituted with different variable regions, e.g. anti-NKp46 variable regions or CDRs of a polypeptide chain sequence disclosed herein are substituted by respective anti-NKp30 variable regions or CDRs, anti-NKp46 variable regions or CDRs of a polypeptide chain sequence disclosed herein are substituted by respective anti-NKG2D variable regions or CDRs. In one embodiment, anti-NKp46 variable regions or CDRs of a polypeptide chain sequence disclosed herein are included for computing identity and the variable regions or CDRs for the antigen binding domain that binds the other antigen (e.g. cancer antigens) are excluded from the sequences that are considered for computing identity.

In one embodiment of any of the polypeptides described herein, the multispecific polypeptide is capable of directing NKp46-expressing NK cells to lyse a target cell of interest (e.g. a target cell expressing an antigen (other than NKp46 or CD16) bound by the multispecific protein).

In one aspect of any of the embodiments herein, provided is a recombinant nucleic acid encoding a first polypeptide chain, and/or a second polypeptide chain and/or a third polypeptide chain of any of the proteins of the disclosure. In one aspect of any of the embodiments herein, provided is a recombinant host cell comprising a nucleic acid encoding a first polypeptide chain, and/or a second polypeptide chain and/or a third polypeptide chain of any of the proteins of the disclosure, optionally wherein the host cell produces a protein of the disclosure with a yield (final productivity, following purification) of at least 1, 2, 3 or 4 mg/L. Also provided is a kit or set of nucleic acids comprising a recombinant nucleic acid encoding a first polypeptide chain of the disclosure, a recombinant nucleic acid encoding a second polypeptide chain of the disclosure, and, optionally, a recombinant nucleic acid encoding a third polypeptide chain of the disclosure. Also provided are methods of making monomeric, heterodimeric and heterotrimeric proteins of the disclosure.

In one embodiment, the invention provides methods of making a heterodimeric protein (e.g. any heterodimeric protein described herein), comprising:

a) providing a first nucleic acid encoding a first polypeptide chain described herein;

b) providing a second nucleic acid encoding a second polypeptide chain described herein;

c) optionally, providing a third nucleic acid encoding a third polypeptide chain described herein; and

d) expressing said first and second (and optionally third) nucleic acids in a host cell to produce a protein comprising said first and second polypeptide (and optionally third) chains, respectively; and recovering a heterodimeric (or optionally a heterotrimeric) protein. Optionally, the heterodimeric (or heterotrimeric) protein produced represents at least 20%, 25% or 30% of the total multispecific proteins obtained prior to purification. Optionally step (d) comprises loading the protein produced onto an affinity purification support, optionally an affinity exchange column, optionally a Protein-A support or column, and collecting the heterodimeric protein; and/or loading the protein produced (e.g., the protein collected following loading onto an affinity exchange or Protein A column) onto an ion exchange column; and collecting the heterodimeric fraction.

In one aspect, provided is a pharmaceutical composition comprising a compound or composition described herein, and a pharmaceutically acceptable carrier.

In one aspect provided is the use of a polypeptide or composition of any one of the above claims as a medicament for the treatment of disease.

In one aspect provided is a method of treating a disease in a subject comprising administering to the subject a compound or composition described herein.

In one embodiment, the disease is a cancer or an infectious disease.

Any of the methods can further be characterized as comprising any step described in the application, including notably in the “Detailed Description of the Invention”). The invention further relates to a protein obtainable by any of present methods. The disclosure further relates to pharmaceutical or diagnostic formulations of the antibodies of the present invention. The disclosure further relates to methods of using antibodies in methods of treatment or diagnosis.

These and additional advantageous aspects and features of the invention may be further described elsewhere herein.

### BRIEF DESCRIPTION OF THE FIGURES

FIGS. 1A, 1B and 1C show structures of exemplary heterotrimeric multispecific protein. FIG. 1A (top panel of FIG. 1) shows a F18 format protein. FIG. 1B (middle panel of FIG. 1) shows a F19 format protein. FIG. 1C (bottom panel of FIG. 1) shows a F20 format protein.

FIG. 2 shows the ability of CD19×NKp46 bispecific proteins having the structure of F18, F19 and F20 formats to mediate Daudi tumor target cell lysis by NK cells as assessed in a classical 4-h ⁵¹Cr-release assay in U-bottom 96 well plates, compared with the most potent proteins known (having the F5 structure). The F18, F19 and F20 were highly potent in mediating target cell lysis, comparable to F5 proteins, with an EC₅₀ of about 1 picomolar.

FIG. 3 shows a comparison of CD19-F18-NKp46-1, CD19-F19-NKp46-1 and CD19-F20-NKp46-1, and full-length anti-CD19 antibodies which engage CD16 (but not NKp46). Each of the F18, F19 and F20 proteins were highly potent in inducing NK cell mediated lysis of tumor target cells, and all were more potent than the full-length anti-CD19 antibody.

FIG. 4 shows a comparison of CD20-F18-NKp46-1, CD20-F19-NKp46-1 and CD20-F20-NKp46-1, and full-length anti-CD20 antibodies which engage CD16 (but not NKp46). Each of the F18, F19 and F20 proteins were highly potent in inducing NK cell mediated lysis of tumor target cells, and all were more potent than the full-length anti-CD20 antibody.

FIG. 5 shows a comparison of the bivalent binding CD20-F20-NKp46-1 protein and the highly potent monovalent binding CD20-F5-NKp46-1 protein, as well as full-length anti-CD20 antibodies which engage CD16 (but not NKp46). F5 and F20 proteins were similarly potent in inducing NK cell mediated lysis of tumor target cells.

FIG. 6 shows a comparison of bivalent binding F20 proteins specific for either CD19 or CD20. Both F20 proteins are far more potent than bivalent binding full-length anti-CD19 or CD20 IgG1 antibodies, and the CD20-F20-NKp46-1 is somewhat more potent than CD19-F20-NKp46-1 protein in inducing target cell lysis of Daudi tumor cells. The full-length anti-CD20 antibodies were more potent that the full-length anti-CD19 IgG1 antibodies.

FIG. 7 shows a comparison of the bivalent binding CD20-F20-NKp46-1 protein and the CD19-CD20-F20-NKp46-1 protein. Surprisingly, despite the anti-CD19 full-length antibodies having lower activity (see FIG. 6) and the CD19-F20-NKp46-1 protein having lower potency that its anti-CD20 equivalent, the CD19-CD20-F20-NKp46-1 protein that binds monovalently to CD19 and monovalently to CD20 showed a dramatic increase in potency (between 1 and 2-log increase in potency based on EC₅₀ values) in mediating Daudi tumor cell lysis than the CD20-F5-NKp46-1 protein.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, more optionally by “consisting of”.

As used herein, the term “antigen binding domain” or “ABD” refers to a domain comprising a three-dimensional structure capable of immunospecifically binding to an epitope. Thus, in one embodiment, said domain can comprise a hypervariable region, optionally a VH and/or VK domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)₂, F(ab′)₂, F(ab)₃, Fv (typically the VK and VH domains of a single arm of an antibody), single-chain Fv (scFv), dsFv, Fd fragments (typically the VH and CH1 domain), and dAb (typically a VH domain) fragments; VH, VK, VhH, and V-NAR domains; minibodies, diabodies, triabodies, tetrabodies, and kappa bodies (see, e.g., I1 et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

The term “antibody derivative”, as used herein, comprises a full-length antibody or a fragment of an antibody, e.g. comprising at least antigen-binding or variable regions thereof, wherein one or more of the amino acids are chemically modified, e.g., by alkylation, PEGylation, acylation, ester formation or amide formation or the like. This includes, but is not limited to, PEGylated antibodies, cysteine-PEGylated antibodies, and variants thereof.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes. By “constant light chain” or “light chain constant region” as used herein is meant the region of an antibody encoded by the kappa (CK) or lambda (CA) light chains. The constant light chain typically comprises a single domain, and as defined herein refers to positions 108-214 of CK, or CA, wherein numbering is according to the EU index (Kabat et al., 1991, Sequences of Proteins of Immunological Interest, 5th Ed., United States Public Health Service, National Institutes of Health, Bethesda). By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant the polypeptide that comprises the VH, CH1, VK, and CL immunoglobulin domains. Fab may refer to this region in isolation, or this region in the context of a polypeptide, multispecific polypeptide or ABD, or any other embodiments as outlined herein.

By “single-chain Fv” or “scFv” as used herein are meant antibody fragments comprising the VH and VK domains of an antibody, wherein these domains are present in a single polypeptide chain. Generally, the Fv polypeptide further comprises a polypeptide linker between the VH and VK domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the VK and VH domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides include but are not limited to antibodies, Fc fusions and Fc fragments. Also, Fc regions according to the invention include variants containing at least one modification that alters (enhances or diminishes) an Fc associated effector function. Also, Fc regions according to the invention include chimeric Fc regions comprising different portions or domains of different Fc regions, e.g., derived from antibodies of different isotype or species.

By “variable region” as used herein is meant the region of an antibody that comprises one or more Ig domains substantially encoded by any of the VK (including VK and VA) and/or VH genes that make up the light chain (including K and A) and heavy chain immunoglobulin genetic loci respectively. A light or heavy chain variable region (VK and VH) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “specifically binds to” means that an antibody or polypeptide can bind preferably in a competitive binding assay to the binding partner, as assessed using either recombinant forms of the proteins, epitopes therein, or native proteins present on the surface of isolated target cells. Competitive binding assays and other methods for determining specific binding are further described below and are well known in the art.

The term “affinity”, as used herein, means the strength of the binding of an antibody or polypeptide to an epitope. The affinity of an antibody is given by the dissociation constant K_{D}, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device).

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “Identity” or “Identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

An “Isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

In the context herein, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

The term “internalization”, used interchangeably with “intracellular intemalization”, refers to the molecular, biochemical and cellular events associated with the process of translocating a molecule from the extracellular surface of a cell to the intracellular surface of a cell. The processes responsible for intracellular intemalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “Inducing and/or increasing internalization” refers to events wherein intracellular internalization is initiated and/or the rate and/or extent of intracellular internalization is increased.

As used herein, the phrase “NK cells” refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell KS and Colonna M). Humana Press. pp. 219-238 (2000).

As used herein, “T cells” refers to a sub-population of lymphocytes that mature in the thymus, and which display, among other molecules T cell receptors on their surface. T cells can be identified by virtue of certain characteristics and biological properties, such as the expression of specific surface antigens including the TCR, CD4 or CD8, the ability of certain T cells to kill tumor or infected cells, the ability of certain T cells to activate other cells of the immune system, and the ability to release protein molecules called cytokines that stimulate or inhibit the immune response. Any of these characteristics and activities can be used to identify T cells, using methods well known in the art. Within the context herein, “active” or “activated” T cells designate biologically active T cells, more particularly T cells having the capacity of cytolysis or of stimulating an immune response by, e.g., secreting cytokines. Active cells can be detected in any of a number of well-known methods, including functional assays and expression-based assays such as the expression of cytokines such as TNF-alpha.

As used herein, an agent that has “agonist” activity at a cell surface receptor (e.g. an activating receptor) is an agent that can cause or increase signalling by the receptor, e.g., an ability of the receptor to activate or transduce an intracellular signaling pathway. Changes in signaling activity can be measured, for example, by assays designed to measure changes in receptor signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of receptor-sensitive promoters and enhancers, or indirectly by a downstream effect mediated by the receptor (e.g. activation of specific cytolytic machinery in NK or T cells). Reporter genes can be naturally occurring genes (e.g. monitoring cytokine production) or they can be genes artificially introduced into a cell. Other genes can be placed under the control of such regulatory elements and thus serve to report the level of receptor signaling.

### Producing Polypeptides

The antigen binding domains (ABDs) described herein can be readily derived from any of a variety of immunoglobulin or non-immunoglobulin scaffolds. Immunoglobulin ABDs can be obtained from variable domains derived from antibodies (from immunoglobulin chains), for example in the form of associated VK and V_{H} domains found on two polypeptide chains, or a single chain antigen binding domain such as a scFv, a V_{H} domain, a VK domain, a dAb, a V-NAR domain or a V_{H}H domain. In certain advantageous proteins formats disclosed herein that directly enable the use of a wide range of variable regions from Fab or scFv without substantial further requirements for pairing and/or folding, the an antigen binding domain (e.g., ABD₁ and ABD₂) can also be readily derived from antibodies as a Fab or scFv. ABD's can also be derived from non-immunoglobulin scaffolds, for example affibodies based on the Z-domain of staphylococcal protein A, engineered Kunitz domains, monobodies or adnectins based on the 10th extracellular domain of human fibronectin III, anticalins derived from lipocalins, DARPins (designed ankyrin repeat domains, multimerized LDLR-A module, avimers or cysteine-rich knottin peptides. See, e.g., Gebauer and Skerra (2009) Current Opinion in Chemical Biology 13:245-255, the disclosure of which is incorporated herein by reference.

Typically, antibodies are initially obtained by immunization of a non-human animal, e.g., a mouse, rat, guinea pig or rabbit, with an immunogen comprising a polypeptide, or a fragment or derivative thereof, typically an immunogenic fragment, for which it is desired to obtain antibodies (e.g. a human polypeptide). The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988), the entire disclosure of which is herein incorporated by reference). Human antibodies may also be produced by using, for immunization, transgenic animals that have been engineered to express a human antibody repertoire (Jakobovitz et Nature 362 (1993) 255), or by selection of antibody repertoires using phage display methods. For example, a XenoMouse (Abgenix, Fremont, Calif.) can be used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. Antibodies may also be produced by selection of combinatorial libraries of immunoglobulins, as disclosed for instance in (Ward et al. Nature, 341 (1989) p. 544, the entire disclosure of which is herein incorporated by reference). Phage display technology (McCafferty et al. (1990) Nature 348:552-553) can be used to produce antibodies from immunoglobulin variable (V) domain gene repertoires from unimmunized donors. See, e.g., Griffith et al. (1993) EMBO J. 12:725-734; U.S. Pat. Nos. 5,565,332; 5,573,905; 5,567,610; and 5,229,275). When combinatorial libraries comprise variable (V) domain gene repertoires of human origin, selection from combinatorial libraries will yield human antibodies.

Additionally, a wide range of antibodies are available in the scientific and patent literature, including DNA and/or amino acid sequences, or from commercial suppliers. Antibodies will typically be directed to a pre-determined antigen. Examples of antibodies include antibodies that recognize an antigen expressed by a target cell that is to be eliminated, for example a proliferating cell or a cell contributing to a disease pathology. Examples include antibodies that recognize tumor antigens, microbial (e.g. bacterial or parasite) antigens or viral antigens.

Variable domains and/or antigen binding domains can be selected based on the desired cellular target, and may include for example cancer antigens, bacterial or viral antigens, etc. As used herein, the term “bacterial antigen” includes, but is not limited to, intact, attenuated or killed bacteria, any structural or functional bacterial protein or carbohydrate, or any peptide portion of a bacterial protein of sufficient length (typically about 8 amino acids or longer) to be antigenic. Examples include gram-positive bacterial antigens and gram-negative bacterial antigens. In some embodiments the bacterial antigen is derived from a bacterium selected from the group consisting of Helicobacter species, in particular Helicobacter pyloris; Borrelia species, in particular Borrelia burgdorferi; Legionella species, in particular Legionella pneumophilia; Mycobactera s species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumonae; anaerobic Streptococcus species; pathogenic Campylobacter species; Enterococcus species; Haemophilus species, in particular Haemophilus influenzae; Bacillus species, in particular Bacillus anthracis; Corynebacterum species, in particular Corynebacterium diphtherae; Erysipelothrix species, in particular Erysipelothrx rhusiopathiae; Clostridium species, in particular C. perfringens, C. tetani; Enterobacter species, in particular Enterobacter aerogenes, Klebsiella species, in particular Klebsiella 1S pneumoniae, Pasteurella species, in particular Pasteurella multocida, Bacteroides species; Fusobacterium species, in particular Fusobacterum nucleatum; Streptobacillus species, in particular Streptobacillus moniliformis; Treponema species, in particular Treponema pertenue; Leptospira; pathogenic Escherichia species; and Actinomyces species, in particular Actinomyces israeli.

As used herein, the term “viral antigen” includes, but is not limited to, intact, attenuated or killed whole virus, any structural or functional viral protein, or any peptide portion of a viral protein of sufficient length (typically about 8 amino acids or longer) to be antigenic. Sources of a viral antigen include, but are not limited to viruses from the families: Retroviridae (e.g., human immunodeficiency viruses, such as HIV-1 (also referred to as HTLV-Ill, LAV or HTLV-Ill/LAV, or HIV-Ill; and other isolates, such as HIV-LP; Picomavirdae (e.g., polio viruses, hepatitis A virus; enteroviruses, human Coxsackie viruses, rhinoviruses, echoviruses); Calciviridae (e.g., strains that cause gastroenteritis); Togavirdae (e.g., equine encephalitis viruses, rubella viruses); Flaviviridae (e.g., dengue viruses, encephalitis viruses, yellow fever viruses); Coronavirdae (e.g., coronaviruses); Rhabdoviridae (e.g., vesicular stomatitis viruses, rabies viruses); Filovirdae (e.g., Ebola viruses); Paramyxovirdae (e.g., parainfluenza viruses, mumps virus, measles virus, respiratory syncytial virus); Orthomyxovirdae (e.g., influenza viruses); Bunyavirdae (e.g., Hantaan viruses, bunya viruses, phleboviruses and Nairo viruses); Arenavirdae (hemorrhagic fever viruses); Reovirdae (e.g., reoviruses, orbiviruses and rotaviruses); Bornavirdae; Hepadnavirdae (Hepatitis B virus); Parvovirdae (parvoviruses); Papovaviridae (papilloma viruses, polyoma viruses); Adenoviridae (most adenoviruses); Herpesvirdae (herpes simplex virus (HSV) 1 and 2, varicella zoster virus, cytomegalovirus (CMV), herpes virus; Poxviridae (variola viruses, vaccinia viruses, pox viruses); and Iridovirdae (e.g., African swine fever virus); and unclassified viruses (e.g., the agent of delta hepatitis (thought to be a defective satellite of hepatitis B virus), Hepatitis C; Norwalk and related viruses, and astroviruses). Alternatively, a viral antigen may be produced recombinantly.

As used herein, the terms “cancer antigen” and “tumor antigen” are used interchangeably and refer to antigens that are expressed by cancer cells or are expressed by non-malignant cells having a pro-tumoral effect (e.g. immune or other cells that are capable of exerting an immunosuppressive effect), and can thereby be exploited in order treat cancer. Cancer antigens can potentially stimulate apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Still other cancer antigens can be expressed on immune cells capable of contributing to or mediating a pro-tumoral effect, e.g. cell that contributes to immune evasion, a monocyte or a macrophage, optionally a suppressor T cell, regulatory T cell, or myeloid-derived suppressor cell.

The cancer antigens are often normal cell surface antigens which are either over-expressed or expressed at abnormal times, or are expressed by a targeted population of cells. Ideally the target antigen is expressed only on proliferative cells (e.g., tumor cells) or pro-tumoral cells (e.g. immune cells having an immunosuppressive effect). Antigens with high tumor specificity or that are truly tumor specific include in particular viral antigens, antigens encoded by mutant genes (e.g., point mutations, fusion proteins resulting from translocations, for example P53, KRAS, NRAS, BCR-ABL, ETV6-AML1) and cancer germline genes (e.g., MAGE-, GAGE and BAGE-family of tumor antigens antigens, the SSX genes).

However target antigens are in many cases also expressed to some extent on healthy cells (they are tumor-associated antigens rather than tumor-specific antigens) and are selected on the basis of differential expression between proliferative/disease tissue and healthy tissue. Often, antigens having low-tumor specificity are differentiation antigens. Example of cancer antigens (or differentiation antigens or tumor-associated antigens) include: Receptor Tyrosine Kinase-like Orphan Receptor 1 (ROR1), Crypto, CD4, CD20, CD30, CD19, CD38, CD47, Glycoprotein NMB, CanAg, Her2 (ErbB2/Neu), a Siglec family member, for example CD22 (Siglec2) or CD33 (Siglec3), CD79, CD138, CD171. PSCA, L1-CAM, PSMA (prostate specific membrane antigen), BCMA, CD52, CD56, CD80, CD70, E-selectin, EphB2, Melanotransferrin, Mud 6 and TMEFF2. Examples of cancer antigens also include Immunoglobulin superfamily (IgSF) such as cytokine receptors, Killer-Ig Like Receptor, CD28 family proteins, for example, Killer-Ig Like Receptor 3DL2 (KIR3DL2), B7-H3, B7-H4, B7-H6, PD-L1, IL-6 receptor. Examples also include major histocompatibility complex class I-related chain A and B polypeptides (MICA and MICB), adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), nectins (e.g. nectin-4), proteins of the UL16-binding protein (ULBP) family, proteins of the retinoic acid early transcript-1 (RAET1) family, carcinoembryonic antigen (CEA), T-cell receptor/CD3-zeta chain, anti-Müllerian hormone Type II receptor, delta-like ligand 4 (DLL4), DR5, ROR1 (also known as Receptor Tyrosine Kinase-Like Orphan Receptor 1 or NTRKRI (EC 2.7.10.1), MUC family, VEGF, VEGF receptors, Angiopoietin-2, PDGF, TGF-alpha, EGF, EGF receptor, members of the human EGF-like receptor family, e.g., HER-2/neu, HER-3, HER-4 or a heterodimeric receptor comprised of at least one HER subunit, gastrin releasing peptide receptor antigen, Muc-1, CA125, integrin receptors, av33 integrins, a581 integrins, αIIbß3-integrins, PDGF beta receptor, IL-8 receptor, IL-6 receptor, CSF1R (tumor-associated monocytes and macrophages), α-fetoprotein, E-cadherin, α-catenin, ß-catenin and γ-catenin, GM2 and GD2 gangliosides, although this is not intended to be exhaustive.

In one aspect, the antigen of interest is an antigen (e.g. any one of the antigens listed above) capable of undergoing intracellular internalization, for example when bound by a conventional human IgG1 antibody, either in the presence of absence of Fcγ receptor cells.

In one aspect, the antigen of interest is an antigen that is expressed at significant levels by healthy cells (in addition to pathogenic cells, tumor cells, pro-tumoral cells, etc.).

In one embodiment, the ABD that binds an antigen of interest is derived from (e.g. comprises the hypervariable region of, or comprises one, two, three, four, five or six of the CDRs of) a parental antibody that binds an antigen of interest (e.g. a murine antibody, a human antibody) which, when bound to its antigenic target (the antigen of interest on cells), increases or induces down-modulation or intracellular internalization of the antigen of interest. In one embodiment, the antigen of interest is a cancer antigen, e.g. one of the cancer antigens listed above known to internalize (e.g. Immunoglobulin superfamily (IgSF) members, for example cytokine receptor α or β chains, Killer-Ig Like Receptors, CD28 family proteins, B7-H3, B7-H4, B7-H6, KIR3DL2, PTK7, ROR1, L1-CAM, Siglec family members, EGF receptor and EGF-like receptor family members, EGFR, HER-2, integrins, anti-Müllerian hormone Type II receptor, CSF-1R, and others). In one embodiment, the antigen target is a polypeptide present on an immune cell capable of mediating a pro-tumoral effect (e.g. by mediating an immunosuppressive effect), for example the immune cell may be a monocyte or a macrophage, optionally a suppressor T cell, regulatory T cell, or myeloid-derived suppressor cell.

In exemplary embodiments, an ABD, variable domain or pair of complementary variable domains will bind an antigen expressed by a target cell that is to be eliminated (e.g., a tumor antigen, microbial (e.g. bacterial or parasitic) antigen, viral antigen, or antigen expressed on an immune cell that is contributing to inflammatory or autoimmune disease, and another ABD, variable domain or pair of complementary variable domains will bind to an antigen expressed on an immune cell, for example an immune effector cell, e.g. a cell surface receptor of an effector cells such as a T or NK cell. Examples of antigens expressed on immune cells, optionally immune effector cells, include antigens expressed on a member of the human lymphoid cell lineage, e.g. a human T cell, a human B cell or a human natural killer (NK) cell, a human monocyte, a human neutrophilic granulocyte or a human dendritic cell. Advantageously, such cells will have a cytotoxic effect on a target cell that is to be eliminated (e.g., that expresses a tumor antigen, microbial antigen, viral antigen, or antigen expressed on an immune cell that is contributing to inflammatory or autoimmune disease). Especially advantageously, the human lymphoid cell is a cytotoxic T cell or NK cell which, when activated, exerts a cytotoxic effect on the target cell. According to this embodiment, then, the cytotoxic activity of the human effector cells is recruited. According to another embodiment, the human effector cell is a member of the human myeloid lineage.

Antigens expressed on an immune cell to which antibodies of fragments that make up multispecific protein can bind also include NK and/or T cell receptors, e.g. any molecule on the surface of NK cells or T cells, respectively, that can serve to direct the NK or T cell to mediate the elimination or lysis of the target cell. Examples include, e.g., members of the immunoglobulin superfamily, members of the killer-cell immunoglobulin-like receptor (KIR) family, the leukocyte immunoglobulin-like receptors (LILR) family, or the lectin family or the NK cell lectin-like receptor family. Activity can be measured for example by bringing target cells and effector cells into contact in presence of the multispecific polypeptide. Optionally the immune cell receptor is an immune effector cell activating receptor, e.g. an activating NK cell or T cell receptor. As used herein, the terms “activating NK cell receptor” and “activating T cell receptor” refers to any molecule on the surface of NK cells or T cells, respectively, that, when stimulated, causes a measurable increase in any property or activity known in the art as associated with NK cell or T cell activity, respectively, such as cytokine (for example IFN-γ or TNF-α) production, increases in intracellular free calcium levels, the ability to lyse target cells in a redirected killing assay as described, e.g. elsewhere in the present specification, or the ability to stimulate NK cell or T cell proliferation, respectively. The term “activating NK receptor” includes but is not limited to DNAX accessory molecule-1 (DNAM-1), 2B4, activating forms of KIR proteins (for example KIR2DS receptors, KIR2DS2, KIR2DS4), NKG2D, NKp30, CD137, CD69, NKp80, NKp44, NKp46, IL-2R, IL-12R, IL-15R, IL-18R and IL-21R. In one embodiment, the activating NK cell receptor is a receptor other than an Fcγ receptor. In one embodiment, the activating NK cell receptor is a receptor other than NKp46.

Activation of cytotoxic T cells may occur via binding of the CD3 antigen as effector antigen on the surface of the cytotoxic T cell by a multispecific (e.g. bispecific) polypeptide of this embodiment. The human CD3 antigen is present on both helper T cells and cytotoxic T cells. Human CD3 denotes an antigen which is expressed on T cells as part of the multimolecular T cell complex and which comprises three different chains: CD3-epsilon, CD3-delta and CD3-gamma. Other effector cell antigens that can be bound by an ABD are the human CD8 antigen, the human CD2 antigen, the human CD28 antigen or the human CD25 antigen.

In one embodiment, the multispecific protein comprises one ABD that binds specifically to an activating receptor present on effector NK or T cells (e.g., NKp46, NKG2D, CD8, CD137, etc.), and two ABDs that each bind to a cancer antigen, wherein the two ABDs that bind to a cancer antigen have a differ in their amino acid sequence and differ in their antigen specificity. Preferably, the two ABDs that each bind to a cancer antigen will bind different cancer antigens, optionally wherein the cancer antigens are known to be co-expressed by a cancer cell (or a cancer promoting cell, such as a cell in tumor tissue or tumor-adjacent tissue), optionally further wherein at least one of the cancer antigens are known to be expressed additionally by healthy (non-cancerous) cells but wherein both cancer antigens are not known to be significantly co-expressed by healthy cells.

“NKp46” refers to a protein or polypeptide encoded by the Ncrl gene or by a cDNA prepared from such a gene. Any naturally occurring isoform, allele, ortholog or variant is encompassed by the term NKp46 polypeptide (e.g., an NKp46 polypeptide 90%, 95%, 98% or 99% identical to SEQ ID NO 1, or a contiguous sequence of at least 20, 30, 50, 100 or 200 amino acid residues thereof). The 304 amino acid residue sequence of human NKp46 (isoform a, NCBI accession number NP_004820) is shown below.

(SEQ ID NO: 1)
MSSTLPALLC VGLCLSQRIS AQQQTLPKPF IWAEPHFMVP
KEKQVTICCQ GNYGAVEYQL HFEGSLFAVD RPKPPERINK
VKFYIPDMNS RMAGQYSCIY RVGELWSEPS NLLDLVVTEM
YDTPTLSVHP GPEVISGEKV TFYCRLDTAT SMFLLLKEGR
SSHVQRGYGK VQAEFPLGPV TTAHRGTYRC FGSYNNHAWS
FPSEPVKLLV TGDIENTSLA PEDPTFPADT WGTYLLTTET
GLQKDHALWD HTAQNLLRMG LAFLVLVALV WFLVEDWLSR
KRTRERASRA STWEGRRRLN TQTL.

The amino acid residue sequence of human NKp30 (NCBI accession number NP_667341) is shown below:

(SEQ ID NO: 2)
MAWMLLLILI MVHPGSCALW VSQPPEIRTL EGSSAFLPCS
FNASQGRLAI GSVTWFRDEV VPGKEVRNGT PEFRGRLAPL
ASSRFLHDHQ AELHIRDVRG HDASIYVCRV EVLGLGVGTG
NGTRLVVEKE HPQLGAGTVL LLRAGFYAVS FLSVAVGSTV
YYQGKCLTWK GPRRQLPAVV PAPLPPPCGS SAHLLPPVPG G.

The amino acid residue sequence of human NKG2D (NCBI accession number CAA04925) is shown below:

(SEQ ID NO: 3)
MGWIRGRRSR HSWEMSEFHN YNLDLKKSDF STRWQKQRCP
VVKSKCRENA SPFFFCCFIA VAMGIRFIIM VAIWSAVFLN
SLFNQEVQIP LTESYCGPCP KNWICYKNNC YQFFDESKNW
YESQASCMSQ NASLLKVYSK EDQDLLKLVK SYHWMGLVHI
PTNGSWQWED GSILSPNLLT IIEMQKGDCA LYASSFKGYI
ENCSTPNTYI CMQRTV.

Examples of ABDs that bind NKp46 include human and humanized VH and VL domains (or the respective Kabat CDR1, 2 and 3 thereof) disclosed in PCT patent publication no.: WO2017/114694, the disclosure of which is incorporated herein by reference. Exemplary VH and VL domains are also shown in Table 1 below.

TABLE 1
anti-NKp46 VH and VL

| | SEQ ID | |
|---|---|---|
| Antibody | NO | Amino acid sequence |
| NKp46-1 VH | 4 | QVQLQQSGPELVKPGASVKMSCKASGYTFTDYVINWGKQRSGQG |
| | | LEWIGEIYPGSGTNYYNEKFKAKATLTADKSSNIAYMQLSSLTSEDS |
| | | AVYFCARRGRYGLYAMDYWGQGTSVTVSS |
| NKp46-1 VL | 5 | DIQMTQTTSSLSASLGDRVTISCRASQDISNYLNWYQQKPDGTVKL |
| | | LIYYTSRLHSGVPSRFSGSGSGTDYSLTINNLEQEDIATYFCQQGNT |
| | | RPWTFGGGTKLEIK |
| NKp46-2 VH | 6 | EVQLQESGPGLVKPSQSLSLTCTVTGYSITSDYAWNWIRQFPGNKL |
| | | EWMGYITYSGSTSYNPSLESRISITRDTSTNQFFLQLNSVTTEDTAT |
| | | YYCARGGYYGSSWGVFAYWGQGTLVTVSA |
| NKp46-2 VL | 7 | DIQMTQSPASLSASVGETVTITCRVSENIYSYLAWYQQKQGKSPQL |
| | | LVYNAKTLAEGVPSRFSGSGSGTQFSLKINSLQPEDFGSYYCQHHY |
| | | GTPWTFGGGTKLEIK |
| NKp46-3 VH | 8 | EVQLQQSGPELVKPGASVKISCKTSGYTFTEYTMHWVKQSHGKSL |
| | | EWIGGISPNIGGTSYNQKFKGKATLTVDKSSSTAYMELRSLTSEDSA |
| | | VYYCARRGGSFDYWGQGTTLTVSS |
| NKp46-3 VL | 9 | DIVMTQSPATLSVTPGDRVSLSCRASQSISDYLHWYQQKSHESPRL |
| | | LIKYASQSISGIPSRFSGSGSGSDFTLSINSVEPEDVGVYYCQNGHS |
| | | FPLTFGAGTKLELK |
| NKp46-4 VH | 10 | QVQLQQSAVELARPGASVKMSCKASGYTFTSFTMHWVKQRPGQG |
| | | LEWIGYINPSSGYTEYNQKFKDKTTLTADKSSSTAYMQLDSLTSDD |
| | | SAVYYCVRGSSRGFDYWGQGTLVTVSA |
| NKp46-4 VL | 11 | DIQMIQSPASLSVSVGETVTITCRASENIYSNLAWFQQKQGKSPQLL |
| | | VYAATNLADGVPSRFSGSGSGTQYSLKINSLQSEDFGIYYCQHFW |
| | | GTPRTFGGGTKLEIK |
| NKp46-6 VH | 12 | QVQLQQPGSVLVRPGASVKLSCKASGYTFTSSWMHWAKQRPGQ |
| | | GLEWIGHIHPNSGISNYNEKFKGKATLTVDTSSSTAYVDLSSLTSED |
| | | SAVYYCARGGRFDDWGAGTTVTVSS |
| NKp46-6 VL | 13 | DIQMTQSPSSLSASLGERVSLTCRASQDIGSSLNWLQQEPDGTIKR |
| | | LIYATSSLDSGVPKRFSGSRSGSDYSLTISSLESEDFVDYYCLQYAS |
| | | SPWTFGGGTKLEIK |
| NKp46-9 VH | 14 | DVQLQESGPGLVKPSQSLSLTCTVTGYSITSDYAWNWIRQFPGNKL |
| | | EWMGYITYSGSTNYNPSLKSRISITRDTSKNQFFLQLNSVTTEDTAT |
| | | YYCARCWDYALYAMDCWGQGTSVTVSS |
| NKp46-9 VL | 15 | DIQMTQSPASLSASVGETVTITCRTSENIYSYLAWCQQKQGKSPQL |
| | | LVYNAKTLAEGVPSRFSGSGSGTHFSLKINSLQPEDFGIYYCQHHY |
| | | DTPLTFGAGTKLELK |

Examples of ABDs that bind NKG2D include VH and VL domains (or the respective Kabat CDR1, 2 and 3 thereof) disclosed in PCT patent publication no.: WO2016/134371, the disclosure of which is incorporated herein by reference. Examples of ABDs that bind NKp30 include VH and VL domains (or the respective Kabat CDR1, 2 and 3 thereof) of the antibodies disclosed in PCT patent publication no.: WO01/36630, the disclosure of which is incorporated herein by reference.

The ABDs or variable domains which are incorporated into the polypeptides can be tested for any desired activity prior to inclusion in a polypeptide. Once appropriate antigen binding domains having desired specificity and/or activity are identified, DNA encoding each variable domain can be placed, in suitable arrangements, in an appropriate expression vector(s), together with DNA encoding any elements such CH1, CK and/or Fc domains and any other optional elements (e.g. DNA encoding a linking sequences) for transfection into an appropriate host(s). The host is then used for the recombinant production of the polypeptide chains that make up the multispecific protein.

An ABD or variable region derived from an antibody will generally comprise at minimum a hypervariable region sufficient to confer binding activity when present in the multimeric polypeptide. It will be appreciated that an ABD or variable region may comprise other amino acids or functional domains as may be desired, including but not limited to linker elements (e.g. linker peptides, constant domain derived sequences, hinges, or fragments thereof, each of which can be placed between a variable domain and a CH1, Cx, CH2 or CH3 domain, or between other domains as needed).

In any embodiment, ABDs or variable regions can be obtained from a humanized antibody in which residues from a complementary-determining region (CDR) of a human antibody are replaced by residues from a CDR of the original antibody (the parent or donor antibody, e.g. a murine or rat antibody) while maintaining the desired specificity, affinity, and capacity of the original antibody. The CDRs of the parent antibody, some or all of which are encoded by nucleic acids originating in a non-human organism, are grafted in whole or in part into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. An antigen binding domain can thus have non-human hypervariable regions or CDRs and human frameworks region sequences (optionally with back mutations).

Polypeptide chains will be arranged in one or more expression vectors so as to produce the polypeptides having the desired domains operably linked to one another. The host cell may be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof.

The polypeptide can then be produced in an appropriate host cell or by any suitable synthetic process and brought into contact under appropriate conditions for the multimeric (e.g. dimer or trimer) polypeptide to form.

Polypeptide Configurations

An isolated hetero-multimeric protein that binds a first and second (and optionally third) antigen of interest can be prepared according to different configurations, in each case involving at least a central (first) polypeptide chain and a further polypeptide chain that are configured to associate by CH1-Cκ heterodimerization and by CH3-CH3 dimerization to form the desired heteromultimeric protein. The protein can be configured to comprise yet a further polypeptide chain. A heterotrimeric protein in which the chains associate preferentially can be produced, in which a first polypeptide chain associates with a second polypeptide chain by CH1-Cκ heterodimerization and by CH3-CH3 dimerization, and with a third polypeptide chain by CH1-Cκ heterodimerization and VH-VK association. The heterodimerization of the first and third chain thus forms an antigen binding domain that has Fab-type structure which has the advantage of avoiding or minimizing presence of non-natural or non-immunoglobulin derived amino acid domains (e.g. non-Ig domains) that might be recognized as immunogenic by the human immune system.

An exemplary heterotrimeric protein can be composed of a first (central) polypeptide chain comprising a first (central) chain comprising a first CH1 or Cκ domain fused to the N-terminus of an Fc region, the Fc region in turn fused to the N-terminus of a variable (V) domain in turn fused to the N-terminus of a further CH1 or Cκ domain (the V and CH1 or Cκ domains forming a V-(CH1/Cκ) unit);

a second polypeptide chain comprising a variable domain fused to the N-terminus of a CH1 or Cκ domain, wherein the variable domain and CH1 or Cκ domain are selected to be complementary to the variable domain and CH1 or Cκ domain of the first chain, such that the second chain binds to the first chain by CH1-Cκ dimerization and VH-VK association, wherein the VH and VK together form a first antigen binding domain that binds a first antigen of interest; and a third polypeptide chain comprising a CH1 or Cκ domain fused to an Fc region, wherein said CH1 or Cκ domain is selected to be complementary to the first CH1 or Cκ domain of the first chain, such that the third chain binds to the first chain by CH1-C1 dimerization and CH3-CH3 dimerization.

Selecting, in the second chain, a variable domain and CH1 or Cκ constant domain to be complementary to the variable domain and CH1 or Cκ constant domain (V-(CH1/Cκ) unit) of the first chain comprises (a) selecting the V domain of the second chain to be VH if the V domain of the V-(CH1/Cκ) unit of the first chain is a VK, and selecting the V domain of the second chain to be VK if the V domain of the V-(CH1/Cκ) unit of the first chain is a VH, and (b) selecting the constant domain of the second chain to be CH1 if the constant domain of the V-(CH1/Cκ) unit of the first chain is a Cκ, and selecting the constant domain of the second chain to be Cκ if the constant domain of the V-(CH1/Cκ) unit of the first chain is a CH1.

Selecting, in the third chain, the CH1 or Cκ domain that is complementary to the first CH1 or Cκ domain of the first chain comprises selecting a CH1 if the constant domain fused to the N-terminus of an Fc region of the first chain is a Cκ, and selecting a Cκ if the constant domain fused to the N-terminus of an Fc region of the first chain is a CH1.

The second polypeptide chain will associate with the first/central polypeptide chain by CH1-Cκ heterodimerization, forming non-covalent interactions and optionally further interchain disulfide bonds between respective hinge domains and between complementary CH1 and CK domains, driving a preferential pairing multimeric polypeptide so long as CH/Cκ and VH/VK domains are chosen to give rise to a preferred dimerization configuration. The resulting Fab-type structure from the dimerization of the two chains thus comprises a VH-VK pair forming an antigen binding domain, and CH1-Cκ pair. In certain configurations, these V-(CH1/Cκ) units of first and second polypeptides can each comprise a non-naturally occurring VH-Cκ or VK-CH1 domain arrangement. In one example, the first chain comprises a VH-Cκ and the second chain comprises a VK-CH1.

The resulting protein can thus be composed of a first (central) polypeptide chain comprising a first CH1 or Cκ domain fused to the N-terminus of an Fc region, the Fc region in turn fused to the N-terminus of a VH domain in turn fused to the N-terminus of a Cκ domain; a second polypeptide chain comprising a VK domain fused to the N-terminus of a CH1 domain, wherein the second chain binds to the first chain by CH1-Cκ dimerization and VH-VK association, wherein the VH and VK together form a first antigen binding domain that binds a first antigen of interest; and a third polypeptide chain comprising a CH1 or Cκ domain fused to an Fc region, wherein said CH1 or Cκ domain is selected to be complementary to the first CH1 or Cκ domain of the first chain, such that the third chain binds to the first chain by CH1-Cκ dimerization and CH3-CH3 dimerization.

In one example, the first chain comprises a CH1 domain fused to the N-terminus of the Fc domain. The resulting protein can thus for example be composed of a first polypeptide chain comprising a CH1 domain fused to the N-terminus of an Fc region, the Fc region in turn fused to the N-terminus of a VH domain in turn fused to the N-terminus of a Cκ domain; a second polypeptide chain comprising a VK domain fused to the N-terminus of a CH1 domain, wherein the second chain binds to the first chain by CH1-Cκ dimerization and VH-VK association, wherein the VH and VK together form a first antigen binding domain that binds a first antigen of interest; and a third polypeptide chain comprising a Cκ domain fused to an Fc region, wherein the third chain binds to the first chain by CH1-Cκ dimerization and CH3-CH3 dimerization.

In any of the embodiments, the first and/or third polypeptide chains can further comprise a single chain antigen binding domain (ABD) positioned N-terminal to the N-terminal (or first) CH1 or Cκ domain. Such ABD(s) can for example be independently composed of an VH and VK pair arranged in a single polypeptide chain, e.g. an scFv. The ABD(s) can alternatively be independently a binding domain obtained or derived from a natural and/or non-antibody ligand (e.g. a soluble ligand, an extracellular domain portion of cell surface receptor) of the antigen of interest to which the ABD is designed to bind. Consequently, the multimeric protein can comprise two or three ABDs, permitting binding to a second and/or third antigen of interest (in addition to the ABD formed by the association of the VH and VK of the first and second chains, which binds to a first antigen of interest). In preferred embodiments, the first antigen of interest is an activating receptor on an immune cell, and the second and/or third antigens of interest are antigens (e.g. cancer antigens) expressed (or co-expressed) by a target cell to be eliminated. The ABD(s) can comprise any amino acid sequence or protein domain that binds an antigen of interest. In one example, an N-terminal ABD(s) comprises a VH and VK domain pair that associate to form an ABD, e.g. as can be readily derived from parental antibody. The VH and VK can thus be placed on a single polypeptide chain and separated from one another by a peptide linker (e.g. comprising G sequence, wherein is an integer, optionally 2, 3 or 4). In one embodiment, the VK domain is fused, via linker sequence, to the N-terminus of the VH domain, which is in turn fused to the N-terminus of the N-terminal CH or CK domain of the respective first and/or third polypeptide chain.

Examples of domain arrangements (from N-to C-terminus) for the first polypeptide for use in constructing a heterotrimer protein include:

VK-VH-CH1-Fc domain-CH1-VK
VH-VK-CH1-Fc domain-CH1-VK
VK-VH-C_{κ}-Fc domain-CH1-VK
VH-VK-C_{κ}-Fc domain-CH1-VK
VK-VH-CH1-Fc domain-C_{κ}-VK
VH-VK-CH1-Fc domain-C_{κ}-VK
VK-VH-C_{κ}-Fc domain-C_{κ}-VK
VH-VK-C_{κ}-Fc domain-C_{κ}-VK
VK-VH-CH1-Fc domain-CH1-VH
VH-VK-CH1-Fc domain-CH1-VH
VK-VH-C_{κ}-Fc domain-CH1-VH
VH-VK-C_{κ}-Fc domain-CH1-VH
CH1-Fc domain-CH1-VK
C_{κ}-Fc domain-CH1-VK
CH1-Fc domain-C_{κ}-VK
C_{κ}-Fc domain-C_{κ}-VK
CH1-Fc domain-CH1-VH
C_{κ}-Fc domain-CH1-VH

Furthermore, any of the first polypeptides (e.g. a polypeptide of the above table) can further comprise a scFv that bind to a further antigen of interest fused to its C-terminus. In one embodiment, the further scFv binds an effector cell (e.g., NK cell, T cell) activating receptor, optionally a receptor other than NKp46.

Examples of domain arrangements (from N- to C-terminus) for the second polypeptide for use in constructing a heterotrimer protein include:

VH-CH1
VH-C_{κ}
VK-CH1
VK-C_{κ}

Furthermore, any of the second polypeptides (e.g. a polypeptide of the above table) can further comprise a scFv that bind to a further antigen of interest fused to its C-terminus.

In the domain arrangements above, the scFv moiety can be characterized as comprising a VH and a VL domain separated by a flexible linker peptide, e.g. having a structure VH-linker-VK or VK-linker-VH.

Examples of domain arrangements (from N- to C-terminus) for the third polypeptide for use in constructing a heterotrimer protein include:

VK-VH-CH1-Fc domain
VH-VK-CH1-Fc domain
VK-VH-C_{κ}-Fc domain
VH-VK-C_{κ}-Fc domain
CH1-Fc domain
C_{κ}-Fc domain

The Fc domain of the first and/or third polypeptide chains may be a full Fc domain (CH2-CH3) or a portion thereof sufficient to confer the desired functionality (e.g. binding to human FcRn and optionally further other human Fc receptors, CD16A).

Heterotrimeric proteins can for example be formed by using a central (first) polypeptide chain comprising, from N- to C-terminus, a first CH1 constant region, an Fc domain, and a VH fused to a Cκ constant region, e.g., comprising the domain arrangement:

CH1-Fc domain-VH-Cκ.

A second polypeptide chain can then comprise a VK domain fused to a CH1 constant region, e.g., comprising the domain arrangement (N- to C-terminal):

VK-CH1.

A third polypeptide chain can comprise from N- to C-terminus, a Cκ constant region and an Fc domain, e.g., comprising the domain arrangement (N- to C-terminal):

Cκ-Fc domain

such that the Cκ of the first chain dimerizes with the CH1 of the second chain, and the VH of the first chain and the VK of the second chain form an antigen binding domain, and such that the CH1 of the first chain and the Cκ of the third chain dimerize, and the CH3 domains of the respective Fc domains of the first and third chains associate. The structures above are in simplified form, and the various chain may comprise additional domains; for example the first and/or third chain can comprise one or more ABD(s) positioned at the N-terminus of the chains (e.g., an ABD that is made up of a VH-VL pair, such as an scFv); for example the second chain may comprise an ABD positioned at the C-terminus of the chain (e.g., a VH-VL pair such as in an scFv).

An example of a configuration of a resulting heterotrimer with a dimeric Fc domain has a domain arrangement, from N- to C-terminus:

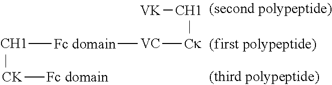

Another example of a configuration of a resulting heterotrimer with a dimeric Fc domain has a domain arrangement, from N- to C-terminus:

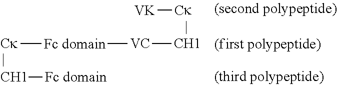

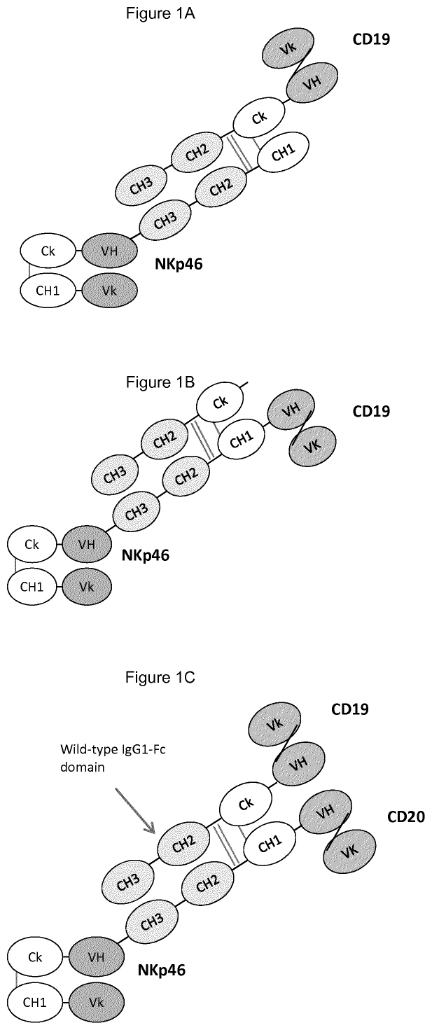

When the first and/or third chains comprise an N-terminal antigen binding domain (to generate a multispecific binding proteins), examples of general structures for domain arrangements of such a resulting heterotrimer protein can comprise, examples of which are also shown in FIGS. 1A, 1B and 1C, from N- to C-terminus:

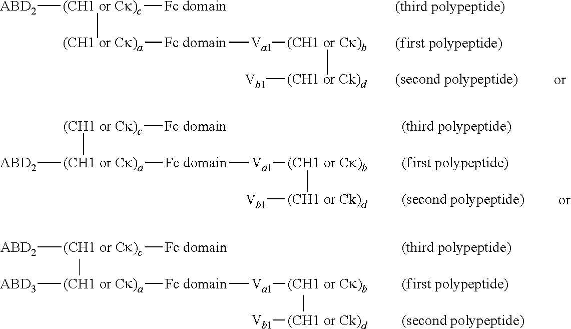

In these structures, the Fc domains of the first and third chains associate via CH3-CH3 dimerization, (CH1 or Cκ)_{c} on the third chain and the (CH1 or Cκ)ₐ. on the central chain undergo CH1-Cκ dimerization, and the (CH1 or Cκ)_{b} on the first chain and the (CH1 or Cκ)_{d} on the second chain undergo CH1-Cκ dimerization. The Vₐ₁ and V_{b1} form a first antigen binding domain (e.g. an ABD₁ that binds an activating receptor on an immune cell). ABD₂ and if present ABD₃ are each self-contained antigen binding domains that can bind an antigen of interest (e.g. a cancer antigen), e.g. without requiring association with a complementary variable domain on a different polypeptide chain. ABD₂ and ABD₃ can for example each comprise a VH and a VK pair (in any desired order), placed on a single chain and separated by a flexible peptide linker (e.g. as an scFv), and a general structure for domain arrangements of such a resulting heterotrimer protein can comprise:

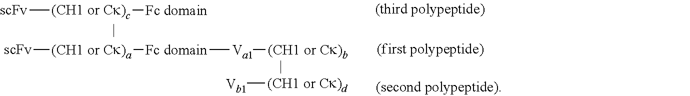

For example, the protein can have the domain arrangement:

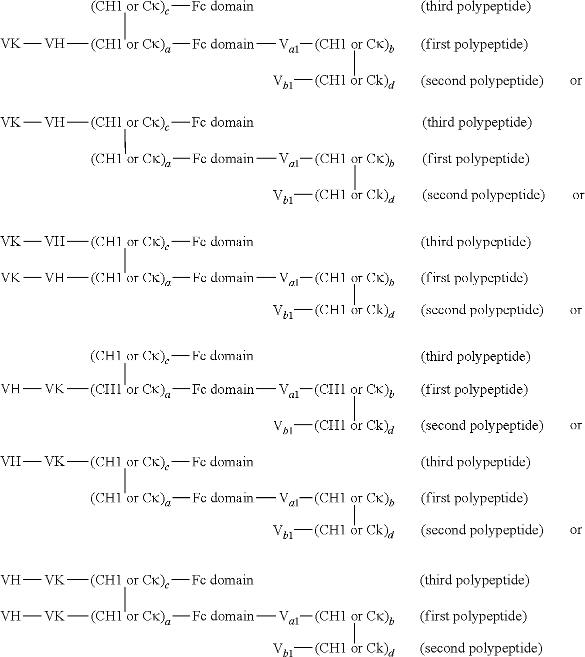

Examples of possible configurations of a resulting heterotrimer with a dimeric Fc domain (some also shown in FIGS. 1A, 1B and 1C) are structures having domain arrangement, from N- to C-terminus:

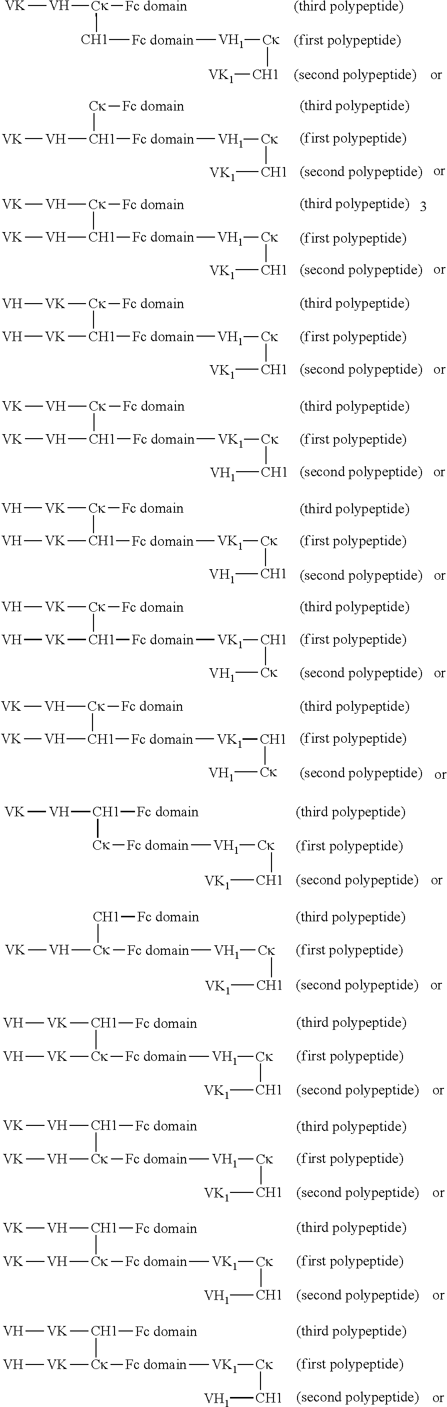

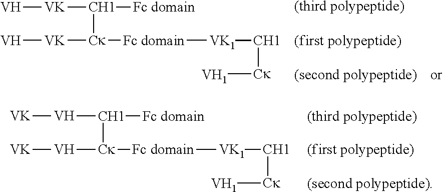

In the structures above, the VH₁ and VK₁ form a first antigen binding domain (e.g. that binds an activating receptor on animmune cell). ABD₂ and if present ABD₃ are represented by the N-terminal VH and VK pairs, that can for example bind an antigen of interest (e.g. a cancer antigen).

The proteins of the above structures can comprise any suitable additional protein domain(s), notably ABDs, if desired. For example, ABDs placed on a single polypeptide chain can be fused to the N- or C-terminus of one or more of the polypeptide chains. In one example, a further ABD is fused to the N-terminus of the first and/or third chain. In one example, a further ABD is fused to the C-terminus of the first or second chain.

An example in which an ABD is fused to the C-terminus of the first chain is shown below:

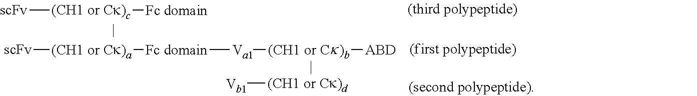

An example in which an ABD is fused to the C-terminus of the second chain is shown below:

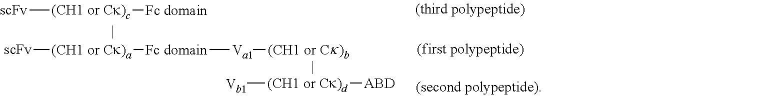

An example in which an ABD is an scFv fused to the C-terminus of the second chain is shown below:

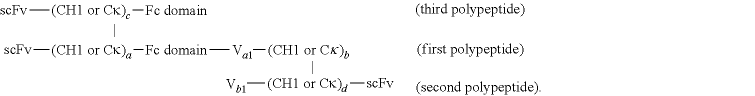

In another aspect, an isolated hetero-multimeric protein that binds a first, second and third antigen of interest can be constructed as a heterodimeric protein. Such a protein can be prepared according to different configurations, in each case involving at least a central (first) polypeptide chain and a second polypeptide chain that are configured to associate by CH1-Cc heterodimerization and by CH3-CH3 dimerization to form the desired heterodimeric protein, similarly as in the heterotrimeric proteins, but wherein the third polypeptide chain is replaced by a VH-VK pair (e.g. as an scFv) placed C-terminal to the Fc domain of the first and/or second polypeptide. A heterodimeric protein in which the chains associate preferentially can thus be produced in which a first polypeptide chain associates with a second polypeptide chain by CH1-Cκ heterodimerization and by CH3-CH3 dimerization, and wherein the first and/or second polypeptide comprise an ABD (e.g. a VH and VK pair that associate to form an ABD) at their N-terminus, and wherein the first and/or second polypeptide further comprise an ABD (e.g. a VH and VK pair that associate to form an ABD) at their C-terminus. In one embodiment the first and second polypeptide each comprise an ABD (e.g. a VH and VK pair that associate to form an ABD) at their N-terminus (e.g. wherein each ABD binds to a different antigen co-expressed by a target cell, for example a tumor cell), and one (but not both) of the first and second polypeptide comprise an ABD (e.g. a VH and VK pair that associate to form an ABD) at its C-terminus (e.g. wherein each ABD binds to an activating receptor on an immune cell).

Examples of domain arrangements (from N- to C-terminus) for the first polypeptide for use in constructing a heterodimer protein include:

VK-VH-CH1-Fc domain-VH-VK
VH-VK-CH1-Fc domain-VH-VK
VK-VH-C_{κ}-Fc domain-VH-VK
VH-VK-C_{κ}-Fc domain-VH-VK
VK-VH-CH1-Fc domain-VK-VH
VH-VK-CH1-Fc domain-VK-VH
VK-VH-C_{κ}-Fc domain-VK-VH
VH-VK-C_{κ}-Fc domain-VK-VH
VK-VH-CH1-Fc domain
VH-VK-CH1-Fc domain
VK-VH-C_{κ}-Fc domain
VH-VK-C_{κ}-Fc domain
VK-VH-CH1-Fc domain
VH-VK-CH1-Fc domain
VK-VH-C_{κ}-Fc domain
VH-VK-C_{κ}-Fc domain

Examples of domain arrangements (from N- to C-terminus) for the second polypeptide for use in constructing a heterodimer protein include the following, wherein if the first polypeptide chain lacks a C-terminal VH/VK pair then the second polypeptide will comprise a C-terminal VH/VK pair:

VK-VH-CH1-Fc domain-VH-VK
VH-VK-CH1-Fc domain-VH-VK
VK-VH-C_{κ}-Fc domain-VH-VK
VH-VK-C_{κ}-Fc domain-VH-VK
VK-VH-CH1-Fc domain-VK-VH
VH-VK-CH1-Fc domain-VK-VH
VK-VH-C_{κ}-Fc domain-VK-VH
VH-VK-C_{κ}-Fc domain-VK-VH
VK-VH-CH1-Fc domain
VH-VK-CH1-Fc domain
VK-VH-C_{κ}-Fc domain
VH-VK-C_{κ}-Fc domain
VK-VH-CH1-Fc domain
VH-VK-CH1-Fc domain
VK-VH-C_{κ}-Fc domain
VH-VK-C_{κ}-Fc domain

The Fc domain of the first and/or second polypeptide chains in the heterodimer may be a full Fc domain (CH2-CH3) or a portion thereof sufficient to confer the desired functionality (e.g. binding to human FcRn and optionally further other human Fc receptors, CD16A).

A resulting exemplary heterodimer protein can comprise the domain arrangement:

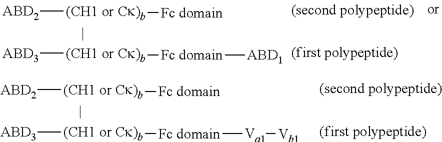

In these structures, the Fc domains of the first and second chains associate via CH3-CH3 dimeization, and (CH1 or Cκ)_{b} on the second chain and the (CH1 or Cκ)ₐ on the first chain undergo CH1-Cκ dimerization. The Vₐ₁ and V_{b1} form a first antigen binding domain (e.g. an ABD₁ that binds an activating receptor on animmune cell). ABD₂ and ABD₃ are each self-contained antigen binding domains that can bind an antigen of interest (e.g. a cancer antigen) without association with a complementary domain on a different polypeptide chain. ABD₂ and ABD₃ can for example each comprise a VH and a VK pair (in any desired order), placed on a single chain and separated by a linking amino acid sequence (e.g. an scFv), and a general structure for domain arrangements of such a resulting heterodimer protein can comprise:

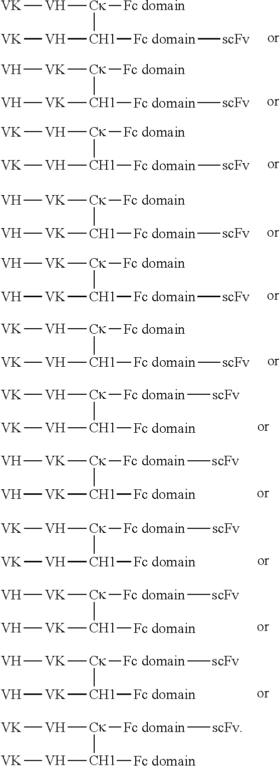

In any embodiment herein, the proteins domains described in the present disclosure can optionally be specified as being described or shown from N- to C-terminal. Protein arrangements of the disclosure for purposes of illustration are shown from N-terminus (on the left) to C-terminus (on the right). Domains can be referred to as fused to one another (e.g. a domain can be specified as being fused to the C-terminus of the domain on its left, and/or a domain can be specified as being fused to the N-terminus of the domain on its right).

Generally, the proteins domains described in the present disclosure can be fused to one another directly or via intervening amino acid sequences, as appropriate given the particular domains to be linked to one another.

Optionally, in any embodiment of any heterodimer or heterotrimer protein, an scFv (e.g. a C-terminal scFv, an N-terminal scFv) can comprise an arrangement, from N- to C-terminal, VH—VK or VK-VH, wherein each variable domain is separated by a peptide linker). In embodiments where a protein comprises a N-terminal scFv on two chains (e.g., in a first and a third chain in a heterotimer), both scFv will preferably share the same domain arrangement to avoid risk of inter-scFv interactions, e.g. both scFv are arranged VH-VK or VK-VH, from N- to C-terminus. When two variable regions that form an antigen binding domain are placed on the same polypeptide chain they are typically linked together by a linker of sufficient length to enable the ABD to fold in such a way as to permit binding to the antigen for which the ABD is intended to bind, e.g., they can form a scFv. Examples of linkers include, for example, linkers comprising glycine and serine residues, for example the VH domain and VK domains of an scFv can be linked together by the amino acid sequence (G₄S)₃.

An Fc domain (e.g. a CH3 domain thereof) can be fused at its C-terminus to the N-terminus of a variable domain via a linker peptide, optionally a linking amino acid sequence comprising or consisting of an amino acid sequence from a naturally occurring in a human protein, optionally in an immunoglobulin (e.g. human immunoglobulin). In one example an Fc domain (e.g. a CH3 domain thereof) is fused at its C-terminus to the N-terminus of a variable domain (VH or VK) via a peptide comprising the amino acid sequence STGS.

Two polypeptide chains will be bound to one another (indicated by “|”) by non-covalent bonds and optionally further by interchain disulfide bonds formed between cysteine residues within complementary CH1 and Cκ domains.

Optionally, in any embodiment herein, a Cκ domain can be replaced by a CA variable domain.

Linkers can be used for fusing a variable region (e.g. a VH or VK) to a CH1 or Cκ domain, or for fusing a C-terminus of a CH3 domain (or C-terminus of an Fc domain) to a variable region. For example, a flexible peptide linker can be used to fuse a VK to a CH1 domain, and/or to fuse a VH domain to a Cκ domain. In one embodiment, a VH domain is fused to a CK domain via a linker peptide comprising the amino sequence RTVA and/or glycine and serine residues followed by the sequence RTVA, for example (G₃S₂)-RTVA. A linker peptide can include a peptide derived from an antibody hinge region.

In one embodiment of the proteins herein, a CH3 domain (or C-terminus of an Fc domain) is fused at its C-terminus to the N-terminus of a variable region (e.g. VH or VK) via a peptide linker (e.g. a flexible peptide linker). In one embodiment, the linker comprises the amino acid sequence STGS. In one embodiment, the linker is a hinge-derived peptide.

Any of the peptide linkers contained in the subject multispecific proteins may comprise a length of at least 4 residues, at least 5 residues, at least 10 residues, at least 15 residues, at least 20 residues, at least 25 residues, at least 30 residues or more. In other embodiments, the linkers comprise a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, between 2-30 residues, between 2 and 50 residues, or between 10 and 50 residues.

In one embodiment, a Cκ domain (e.g., of the first or third polypeptide chain) is linked or fused to the N-terminus of an Fc domain (e.g. to the N-terminus of a CH2 domain of an Fc domain) via a linker peptide, e.g., a peptide that comprises a fragment of a CH1 domain and/or hinge region. For example, a N-terminal amino acid sequence of CH1 can be fused to a variable domain in order to mimic as closely as possible the natural structure of a wild-type antibody. In one embodiment, the linker comprises an amino acid sequence from a hinge domain or an N-terminal CH1 amino acid. The sequence can be, for example, between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, or between 2-30 residues.

In one embodiment, when a Cκ domain of the first or third polypeptide chain is linked or fused at its C-terminus to the N-terminus of an Fc domain (e.g. to the N-terminus of a CH2 domain of an Fc domain) via a linker that comprises a fragment of a CH1 domain and/or hinge region, the complementary CH1 domain of the complementary first of third polypeptide chain will be similarly fused to the N-terminus of an Fc domain (e.g. to the N-terminus of a CH2 domain of an Fc domain) via the same or substantially the same linker that comprises a fragment of a CH1 domain and/or hinge region.

In one embodiment, a CH1 and/or Cκ domain is linked or fused to an Fc domain via a hinge region (or fragment thereof) derived form a hinge domain of a human IgG1 antibody. For example a hinge domain may comprise the amino acid sequence: THTCPPCPAPELL (SEQ ID NO: 27), or an amino acid sequence at least 60%, 70%, 80% or 90% identical thereto, optionally wherein one or both cysteines are deleted or substituted by a different amino acid residue.

In one embodiment, a CH1 and/or Cκ domain is linked or fused to an Fc domain via a hinge region (or fragment thereof) derived form a hinge domain of a human IgG1 antibody. For example a hinge domain may comprise the amino acid sequence: THTCSSCPAPELL (SEQ ID NO: 28), or an amino acid sequence at least 60%, 70%, 80% or 90% identical thereto, optionally wherein one or both cysteines are deleted or substituted by a different amino acid residue.

In one embodiment, the hinge region (or fragment thereof) is derived from a Cμ2-C Cμ3 hinge domain of a human IgM antibody. For example a hinge domain may comprise the amino acid sequence: NASSMCVPSPAPELL (SEQ ID NO: 29), or an amino acid sequence at least 60%, 70%, 80% or 90% identical thereto, optionally wherein one or both cysteines are deleted or substituted by a different amino acid residue.

Polypeptide chains that dimerize and associate with one another via non-covalent bonds may or may not additionally be bound by an interchain disulfide bond formed between respective CH1 and Cκ domains, and/or between respective hinge domains on the chains. CH1, Cκ and/or hinge domains (or other suitable linking amino acid sequences) can optionally be configured such that interchain disulfide bonds are formed between chains such that the desired pairing of chains is favored and undesired or incorrect disulfide bond formation is avoided. For example, when two polypeptide chains to be paired each possess a CH1 or Cκ adjacent to a hinge domain, the polypeptide chains can be configured such that the number of available cysteines for interchain disulfide bond formation between respective CH1/Cκ-hinge segments is reduced (or is entirely eliminated). For example, the amino acid sequences of respective CH1, Cκ and/or hinge domains can be modified to remove cysteine residues in both the CH1/Cκ and the hinge domain of a polypeptide; thereby the CH1 and Cκ domains of the two chains that dimerize will associate via non-covalent interaction(s).

In another example, the CH1 and/or Cκ domain adjacent (e.g., N-terminal to) a hinge domain comprises a cysteine capable of interchain disulfide bond formation, and the hinge domain which is placed at the C-terminus of the CH1 or Cκ comprises a deletion or substitution of one or both cysteines of the hinge (e.g. Cys 239 and Cys 242, as numbered for human IgG1 hinge according to Kabat). In one embodiment, the hinge region (or fragment thereof) comprise the amino acid sequence: THTSPPSPAPELL (SEQ ID NO: 30), or an amino acid sequence at least 60%, 70%, 80% or 90% identical thereto.

In another example, the CH1 and/or Cκ domain adjacent (e.g., N-terminal to) a hinge domain comprises a deletion or substitution at a cysteine residue capable of interchain disulfide bond formation, and the hinge domain placed at the C-terminus of the CH1 or Cκ comprises one or both cysteines of the hinge (e.g. Cys 239 and Cys 242, as numbered for human IgG1 hinge according to Kabat). In one embodiment, the hinge region (or fragment thereof) comprises the amino acid sequence: THTCSSCPAPELL (SEQ ID NO: 28), or an amino acid sequence at least 60%, 70%, 80% or 90% identical thereto.

In another example, a hinge region is derived from an IgM antibody. In such embodiments, the CH1/CK pairing mimics the Cp2 domain homodimerization in IgM antibodies. For example, the CH1 or Cκ domain adjacent (e.g., N-terminal to) a hinge domain comprises a deletion or substitution at a cysteine capable of interchain disulfide bond formation, and an IgM hinge domain which is placed at the C-terminus of the CH1 or Cκ comprises one or both cysteines of the hinge. In one embodiment, the hinge region (or fragment thereof) comprises the amino acid sequence: THTCSSCPAPELL (SEQ ID NO: 28), or an amino acid sequence at least 60%, 70%, 80% or 90% identical thereto.

Constant region domains can be derived from any suitable human antibody, including, the constant heavy (CH1) and light (Cx) domains, hinge domains, CH2 and CH3 domains. “CH1” generally refers to positions 118-220 according to the EU index as in Kabat. “CH2” generally refers to positions 237-340 according to the EU index as in Kabat, and “CH3” generally refers to positions 341-447 according to the EU index as in Kabat.

A “hinge or hinge region” or “antibody hinge region” herein refers to the flexible polypeptide or linker between the first and second constant domains of an antibody. Structurally, the IgG CH1 domain ends at EU position 220, and the IgG CH2 domain begins at residue EU position 237. Thus for an IgG the hinge generally includes positions 221 (D221 in IgG1) to 236 (G236 in IgG1), wherein the numbering is according to the EU index as in Kabat. References to specific amino acid residues within constant region domains found within the polypeptides shall be, unless otherwise indicated or as otherwise dictated by context, be defined according to Kabat, in the context of an IgG antibody.

CH2 and CH3 domains which may be present in the subject antibodies or multispecific proteins can be derived from any suitable antibody. Such CH2 and CH3 domains can be used as wild-type domains or may serve as the basis for a modified CH2 or CH3 domain. Optionally the CH2 and/or CH3 domain is of human origin or may comprise that of another species (e.g., rodent, rabbit, non-human primate) or may comprise a modified or chimeric CH2 and/or CH3 domain, e.g., one comprising portions or residues from different CH2 or CH3 domains, e.g., from different antibody isotypes or species antibodies.

In any of the domain arrangements, the Fc domain may comprise a CH2-CH3 unit (a full length CH2 and CH3 domain or a fragment thereof). The proteins of the disclosure will comprise two chains (e.g. the first and third chains) with Fc domains (the resulting protein has a dimeric Fc domain) comprising a CH3 domain or fragment thereof, wherein the CH3 domain will be capable of undergoing CH3-CH3 dimerization. In one example, the CH3 domain capable of CH3-CH3 dimerization is a wild-type CH3 domain.

In one example, the CH3 domain(s) capable of CH3-CH3 dimerization are modified CH3 domains that enhance preferential CH3-CH3 dimerization. An example of such a modified CH3 is employs a “knob-into-holes” approach in which the CH3 domain interface of the Fc region is mutated, wherein each of two chains have different complementary mutations so that two Fc domain-containing chains preferentially form heterodimers. These mutations create altered charge polarity across the Fc dimer interface such that co-expression of electrostatically matched Fc chains support favorable attractive interactions thereby promoting desired Fc heterodimer formation, whereas unfavorable repulsive charge interactions suppress unwanted Fc homodimer formation. For example one heavy chain comprises a Kabat T366W substitution and the second heavy chain comprises a Kabat T366S, L368A and Y407V substitution, see, e.g. Ridgway et al (1996) Protein Eng., 9, pp. 617-621; Atwell (1997) J. Mol. Biol., 270, pp. 26-35; and WO2009/089004, the disclosures of which are incorporated herein by reference. In another approach, one heavy chain comprises a Kabat F405L substitution and the second heavy chain comprises a Kabat K409R substitution, see, e.g., Labrijn et al. (2013) Proc. Natl. Acad. Sci. U.S.A., 110, pp. 5145-5150. In another approach, one heavy chain comprises Kabat T350V, L351Y, F405A, and Y407V substitutions and the second heavy chain comprises Kabat T350V, T366S, K392L, and T394W substitutions, see, e.g. Von Kreudenstein et al., (2013) mAbs 5:646-654. In another approach, one heavy chain comprises both Kabat K409D and K392D substitutions and the second heavy chain comprises both Kabat D399K and E356K substitutions, see, e.g. Gunasekaran et al., (2010) J. Biol. Chem. 285:19637-19646. In another approach, one heavy chain comprises D221E, P228E and L368E substitutions and the second heavy chain comprises Kabat D221R, P228R, and K409R substitutions, see, e.g. Strop et al., (2012) J. Mol. Biol. 420: 204-219. In another approach, one heavy chain comprises Kabat S364H and F405A substitutions and the second heavy chain comprises Kabat Y349T and, T394F substitutions, see, e.g. Moore et al., (2011) mAbs 3: 546-557. In another approach, one heavy chain comprises a Kabat H435R substitution and the second heavy chain optionally may or may not comprise a substitution, see, e.g. U.S. Pat. No. 8,586,713. In one embodiment of any of the polypeptides or methods herein, the CH3 domain comprises an amino acid substitution at 1, 2, 3, 4, 5, 6 or 7 of the positions L351, T366, L368, P395, F405, T407 (or Y407) and/or K409 (EU numbering as in Kabat).

In embodiments where a multispecific is intended not to bind to human CD16A polypeptide, a CH2 and/or CH3 domain (or Fc domain comprising same) may comprise a modification to decrease or abolish binding to FcγRIIIA (CD16). For example, CH2 mutations in a dimeric Fc domain proteins at reside N297 (Kabat numbering) can eliminate CD16A binding. However the person of skill in the art will appreciate that other configurations can be implemented. For example, substitutions into human IgG1 of IgG2 residues at positions 233-236 and IgG4 residues at positions 327, 330 and 331 were shown to greatly reduce binding to Fcγ receptors and thus ADCC and CDC. Furthermore, Idusogie et al. (2000) J Immunol. 164(8):4178-84 demonstrated that alanine substitution at different positions, including K322, significantly reduced complement activation.

In certain embodiments herein where binding to CD16A is desired, a CH2 and/or CH3 domain (or Fc domain comprising same) may be wild-type domains or may comprise one or more amino acid modifications (e.g. amino acid substitutions) which increase binding to human CD16 and optionally another receptor such as FcRn. Optionally, the modifications will not substantially decrease or abolish the ability of the Fc-derived polypeptide to bind to neonatal Fc receptor (FcRn), e.g. human FcRn. Typical modifications include modified human IgG1-derived constant regions comprising at least one amino acid modification (e.g. substitution, deletions, insertions), and/or altered types of glycosylation, e.g., hypofucosylation. Such modifications can affect interaction with Fc receptors: FcγRI (CD64), FcγRII (CD32), and FcγRIII (CD16). FcγRI (CD64), FcγRIIA (CD32A) and FcγRIII (CD 16) are activating (i.e., immune system enhancing) receptors while FcγRIIB (CD32B) is an inhibiting (i.e., immune system dampening) receptor. A modification may, for example, increase binding of the Fc domain to FcγRIIIa on effector (e.g. NK) cells and/or decrease binding to FcγRIIB. Examples of modifications are provided in PCT publication no. WO2014/044686, the disclosure of which is incorporated herein by reference. Specific mutations in IgG1 which affect (enhance) FcγRIIIa or FcRn binding are also set forth below.

| | | | Effector | Effect of |
|---|---|---|---|---|
| Isotype | Species | Modification | Function | Modification |
| IgG1 | Human | T250Q/M428L | Increased | Increased |
| | | | binding to | half-life |
| | | | FcRn | |
| IgG1 | Human | 1M252Y/S254T/ | Increased | Increased |
| | | T256E + | binding to | half-life |
| | | H433K/N434F | FcRn | |
| IgG1 | Human | E333A | Increased | Increased |
| | | | binding to | ADCC and |
| | | | FcγRIIIa | CDC |
| IgG1 | Human | S239D/A330L/ | Increased | Increased |
| | | I332E | binding to | ADCC |
| | | | FcγRIIIa | |
| IgG1 | Human | P257I/Q311 | Increased | Unchanged |
| | | | binding to | half-life |
| | | | FcRn | |
| IgG1 | Human | S239D/I332E/ | Increased | Increased |
| | | G236A | FcγRIIa/ | macrophage |
| | | | FcγRIIb ratio | phagocytosis |

In some embodiments, the multispecific protein comprises a variant Fc region comprise at least one amino acid modification (for example, possessing 1, 2, 3, 4, 5, 6, 7, 8, 9, or more amino acid modifications) in the CH2 and/or CH3 domain of the Fc region, wherein the modification enhances binding to a human CD16 polypeptide. In other embodiments, the multispecific protein comprises at least one amino acid modification (for example, 1, 2, 3, 4, 5, 6, 7, 8, 9, or more amino acid modifications) in the CH2 domain of the Fc region from amino acids 237-341, or within the lower hinge-CH2 region that comprises residues 231-341. In some embodiments, the multispecific protein comprises at least two amino acid modifications (for example, 2, 3, 4, 5, 6, 7, 8, 9, or more amino acid modifications), wherein at least one of such modifications is within the CH3 region and at least one such modifications is within the CH2 region. Encompassed also are amino acid modifications in the hinge region. In one embodiment, encompassed are amino acid modifications in the CH1 domain, optionally in the upper hinge region that comprises residues 216-230 (Kabat EU numbering). Any suitable functional combination of Fc modifications can be made, for example any combination of the different Fc modifications which are disclosed in any of United States Patents Nos. U.S. Pat. Nos. 7,632,497; 7,521,542; 7,425,619; 7,416,727; 7,371,826; 7,355,008; 7,335,742; 7,332,581; 7,183,387; 7,122,637; 6,821,505 and 6,737,056; and/or in PCT Publications Nos. WO2011/109400; WO 2008/105886; WO 2008/002933; WO 2007/021841; WO 2007/106707; WO 06/088494; WO 05/115452; WO 05/110474; WO 04/1032269; WO 00/42072; WO 06/088494; WO 07/024249; WO 05/047327; WO 04/099249 and WO 04/063351; and/or in Lazar et al. (2006) Proc. Nat. Acad. Sci. USA 103(11):405-410; Presta, L. G. et al. (2002) Biochem. Soc. Trans. 30(4):487-490; Shields, R. L. et al. (2002) J. Biol. Chem. 26; 277(30):26733-26740 and Shields, R. L. et al. (2001) J. Biol. Chem. 276(9):6591-6604).

In some embodiments, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has an enhanced binding affinity for human CD16 relative to the same molecule comprising a wild-type Fc region, optionally wherein the variant Fc region comprises a substitution at any one or more of positions 221, 239, 243, 247, 255, 256, 258, 267, 268, 269, 270, 272, 276, 278, 280, 283, 285, 286, 289, 290, 292, 293, 294, 295, 296, 298, 300, 301, 303, 305, 307, 308, 309, 310, 311, 312, 316, 320, 322, 326, 329, 330, 332, 331, 332, 333, 334, 335, 337, 338, 339, 340, 359, 360, 370, 373, 376, 378, 392, 396, 399, 402, 404, 416, 419, 421, 430, 434, 435, 437, 438 and/or 439 (Kabat EU numbering).

In one embodiment, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has enhanced binding affinity for human CD16 relative to a molecule comprising a wild-type Fc region, optionally wherein the variant Fc region comprises a substitution at any one or more of positions 239, 298, 330, 332, 333 and/or 334 (e.g. S239D, S298A, A330L, 1332E, E333A and/or K334A substitutions), optionally wherein the variant Fc region comprises a substitution at residues S239 and 1332, e.g. a S239D and 1332E substitution (Kabat EU numbering).

When such hetero-multimeric antibodies have Fc regions derived from a human IgG2 or IgG4 that lack or have low natural binding to human Fc receptors, the Fc regions of these antibodies can be engineered to contain amino acid modifications that permit CD16 binding. In some embodiments, the antibody may comprise mammalian antibody-type N-linked glycosylation at residue N297 (Kabat EU numbering).

In some embodiments, the multispecific protein comprises an Fc domain comprising altered glycosylation patterns that increase binding affinity for human CD16. Such carbohydrate modifications can be accomplished by, for example, by expressing a nucleic acid encoding the multispecific protein in a host cell with altered glycosylation machinery. Cells with altered glycosylation machinery are known in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 06/133148; WO 03/035835; WO 99/54342, each of which is incorporated herein by reference in its entirety. In one aspect, the multispecific protein contains one or more hypofucosylated constant regions. Such multispecific protein may comprise an amino acid alteration or may not comprise an amino acid alteration and/or may be expressed or synthesized or treated under conditions that result in hypofucosylation. In one aspect, a multispecific protein composition comprises a multispecific protein described herein, wherein at least 20, 30, 40, 50, 60, 75, 85, 90, 95% or substantially all of the antibody species in the composition have a constant region comprising a core carbohydrate structure (e.g. complex, hybrid and high mannose structures) which lacks fucose. In one embodiment, provided is a multispecific protein composition which is free of antibodies comprising a core carbohydrate structure having fucose. The core carbohydrate will preferably be a sugar chain at Asn297.

Optionally, a multispecific protein comprising a dimeric Fc domain can be characterized by having a binding affinity to a human CD16 polypeptide that is within 1-log of that of a conventional human IgG1 antibody, e.g., as assessed by surface plasmon resonance.

In one embodiment, the multispecific protein comprising a dimeric Fc domain engineered to enhance Fc receptor binding can be characterized by having a binding affinity to a human CD16 polypeptide that is at least 1-log greater than that of a conventional or wild-type human IgG1 antibody, e.g., as assessed by surface plasmon resonance.

Optionally a multispecific protein comprising a dimeric Fc domain can be characterized by a Kd for binding (monovalent) to a human CD16 polypeptide of less than 10⁻⁵ M (10 μmolar), optionally less than 10⁻⁶ M (1 μmolar), as assessed by surface plasmon resonance (e.g. SPR measurements performed on a Biacore T100 apparatus (Biacore GE Healthcare)), with bispecific antibodies immobilized on a Sensor Chip CM5 and serial dilutions of soluble CD16 polypeptide injected over the immobilized bispecific antibodies.

In one embodiment, the disclosure provides methods of making a heterodimeric protein (e.g. any heterodimeric protein described herein), comprising:

a) providing a first nucleic acid encoding a first polypeptide chain described herein, and an Fc domain or portion thereof interposed between the first and second variable domains);

b) providing a second nucleic acid encoding a second polypeptide chain described herein, and

c) expressing said first and second nucleic acids in a host cell to produce a protein comprising said first and second polypeptide chains, respectively; and recovering a heterodimeric protein comprising a dimeric Fc domain. Optionally, the heterodimeric protein produced represents at least 20%, 25% or 30% of the total proteins (e.g. bispecific proteins) prior to purification. Optionally step (c) comprises loading the protein produced onto an affinity purification support, optionally an affinity exchange column, optionally a Protein-A support or column, and collecting the heterodimeric protein; and/or loading the protein produced (or the protein collected following loading onto an affinity exchange or Protein A column) onto an ion exchange column; and collecting the heterodimeric fraction. In one embodiment, the second variable domain (optionally together with the third variable domain) of the first polypeptide chain binds an NK or T cell activating receptor (e.g. NKp46, NKp30, CD137, NKG2D).

In one embodiment, the disclosure provides methods of making a heterotrimeric protein (e.g. any heterotrimeric protein described herein), comprising:

a) providing a first nucleic acid encoding a first polypeptide chain described herein;

b) providing a second nucleic acid encoding a second polypeptide chain described herein;

c) providing a third nucleic acid encoding a third polypeptide chain described herein; and

d) expressing said first, second and third nucleic acids in a host cell to produce a protein comprising said first, second and third polypeptide chains, respectively; and recovering a heterotrimeric protein comprising a dimeric Fc domain. Optionally, the heterotrimeric protein produced represents at least 20%, 25% or 30% of the total proteins (e.g. bispecific proteins) prior to purification. Optionally step (c) comprises loading the protein produced onto an affinity purification support, optionally an affinity exchange column, optionally a Protein-A support or column, and collecting the heterotrimeric protein; and/or loading the protein produced (or the protein collected following loading onto an affinity exchange or Protein A column) onto an ion exchange column; and collecting the heterotrimeric fraction. In one embodiment, the second variable domain (optionally together with the third variable domain) of the first polypeptide chain binds an NK or T cell activating receptor (e.g. NKp46, NKp30, CD137, NKG2D).

By virtue of their ability to be produced in standard cell lines and standardized methods with high yields, unlike BiTE™, DART™ and other bispecific formats, the proteins of the disclosure also provide a convenient tool for screening for the most effective variable regions to incorporate into a multispecific protein, and/or the most effective antigens (e.g., tumor-associated antigen) to target. For example, the proteins can be advantageously used in a method to determine whether a first and a second different candidate tumor-associated antigen (where one or both antigen are known to be expressed on healthy cells in addition to tumor cells) are suitable for targeting by a protein for the treatment of cancer.

In one aspect, the present disclosure provides a method for identifying or evaluating candidate variable regions and/or tumor associated antigens for use in a heteromultimeric (e.g. heterodimeric) protein, comprising the steps of:

a) providing a plurality of nucleic acid pairs, wherein each pair includes one nucleic acid encoding a heavy chain candidate variable region and one nucleic acid encoding a light chain candidate variable region, for each of a plurality of heavy and light chain variable region pairs (e.g., obtained from different antibodies binding the same or different antigen(s) of interest);

b) for each of the plurality nucleic acid pairs, making a heteromultimeric, heterodimeric or heterotrimeric protein of the disclosure (e.g. according to the methods of making a heterodimeric or heterotrimeric protein, above); and

c) evaluating the plurality of heteromultimeric, heterodimeric or heterotrimer proteins produced for a biological activity of interest, e.g., an activity disclosed herein. In this method, the protein binds an activating receptor (e.g. NKp46, NKp30,CD137, NKG2D via one antigen binding domain, and the plurality of nucleic acid pairs encode an VH and VK pair that a cancer antigen. In one embodiment, the plurality of nucleic acid pairs encode a heavy chain and light chain variable regions from a plurality of different antibodies that bind a particular cancer antigen or tumor-associated antigen. In one embodiment, the plurality of nucleic acid pairs encode (i) a heavy chain and light chain variable regions from a first antibody that a first cancer antigen or tumor-associated antigen and (ii) a heavy chain and light chain variable regions from a second antibody that binds a second cancer antigen or tumor-associated antigen, wherein the first and second cancer antigens or tumor-associated antigens are different.

Optionally, the heterodimeric protein produced represents at least 20%, 25% or 30% of the total proteins prior to purification. Optionally the recovering step comprises loading the protein produced onto an affinity purification support, optionally an affinity exchange column, optionally a Protein-A support or column, and collecting the heterotrimeric protein; and/or loading the protein produced (or the protein collected following loading onto a affinity exchange or Protein A column) onto an ion exchange column; and collecting the heterotrimeric fraction.

In one aspect of the any of the embodiments herein, recovering a heterodimeric or heterotrimer protein can comprise introducing the protein to a solid phase so as to immobilize the protein. The immobilized protein can then subsequently be eluted. Generally, the solid support may be any suitable insoluble, functionalized material to which the proteins can be reversibly attached, either directly or indirectly, allowing them to be separated from unwanted materials, for example, excess reagents, contaminants, and solvents. Examples of solid supports include, for example, functionalized polymeric materials, e.g., agarose, or its bead form Sepharose®, dextran, polystyrene and polypropylene, or mixtures thereof; compact discs comprising microfluidic channel structures; protein array chips; pipet tips; membranes, e.g., nitrocellulose or PVDF membranes; and microparticles, e.g., paramagnetic or non-paramagnetic beads. In some embodiments, an affinity medium will be bound to the solid support and the protein will be indirectly attached to solid support via the affinity medium. In one aspect, the solid support comprises a protein A affinity medium or protein G affinity medium. A “protein A affinity medium” and a “protein G affinity medium” each refer to a solid phase onto which is bound a natural or synthetic protein comprising an Fc-binding domain of protein A or protein G, respectively, or a mutated variant or fragment of an Fc-binding domain of protein A or protein G, respectively, which variant or fragment retains the affinity for an Fc-portion of an antibody. Protein A and Protein G are bacterial cell wall proteins that have binding sites for the Fc portion of mammalian IgG. The capacity of these proteins for IgG varies with the species. In general, IgGs have a higher affinity for Protein G than for Protein A, and Protein G can bind IgG from a wider variety of species. The affinity of various IgG subclasses, especially from mouse and human, for Protein A varies more than for Protein G. Protein A can, therefore, be used to prepare isotypically pure IgG from some species. When covalentiy attached to a solid matrix, such as cross-linked agarose, these proteins can be used to capture and purify antigen-protein complexes from biochemical solutions. Commercially available products include, e.g., Protein G, A or L bonded to agarose or sepharose beads, for example EZview Red Protein G Affinity Gel is Protein G covalently bonded to 4% Agarose beads (Sigma Aldrich Co); or POROS® A, G, and CaptureSelect® HPLC columns (Invitrogen Inc.). Affinity capture reagents are also described, for example, in the Antibody Purification Handbook, Biosciences, publication No. 18-1037-46, Edition AC, the disclosure of which is hereby incorporated by reference).

Once the multispecific protein is produced it can be assessed for biological activity. In one aspect of any embodiment herein, where a protein binds an antigen on a target cell to be eliminated and an activating receptor on an effector cell, a multispecific protein is capable of inducing activation of an immune effector cell (e.g. an NK cell, a T cell) when the protein is incubated in the presence of the effector cell and a target cell that expresses the antigen of interest). In one aspect of any embodiment herein, a multispecific protein is capable of inducing signaling at an immune effector cell activating receptor when the protein is incubated in the presence of the effector cell and a target cell that expresses the antigen of interest). Optionally, effector cell activation or signaling in characterized by increased expression of a cell surface marker of activation, e.g. CD107, CD69, etc. Activity can be measured for example by bringing target cells and effector cells into contact with one another, in presence of the multispecific polypeptide. In one example, aggregation of target cells and effector cells is measured. In another example, the multispecific protein may, for example, be assessed for the ability to cause a measurable increase in any property or activity known in the art as associated with NK cell activity, respectively, such as marker of cytotoxicity (CD107) or cytokine production (for example IFN-γ or TNF-α), increases in intracellular free calcium levels, the ability to lyse target cells in a redirected killing assay, etc. In one embodiment of any of the methods of identifying, evaluating or making a protein, the method comprises a step of evaluating the multispecific protein for its ability to induce or increase the activity immune cells that express an activating receptor bound by an ABD of a multispecific protein (e.g. a marker of activation or cytotoxicity, cytokine production, ability to lyse a target cell, etc.), when incubated in the presence of the immune cells and target cells expressing an antigen of interest bound by an ABD of the multispecific protein (e.g. the cancer antigen). In one embodiment, the immune cells express NKp46, CD16 and/or CD137. In one embodiment, the cells are NKp46⁺ NK cells. In one embodiment, the immune cells are CD16⁺ cells. In one embodiment, the immune cells are CD137⁺ cells.

In the presence of target cells (target cells expressing the antigen of interest) and effector cells that express the activating receptor bound by the protein, the multispecific protein will be capable of causing an increase in a property or activity associated with effector (e.g. NK cell, T cell) cell activity (e.g. activation of NK cell cytotoxicity, CD107 expression, IFNγ production) in vitro. For example, a multispecific protein of the disclosure can be selected for the ability to increase an NK or T cell activity by more than about 20%, preferably with at least about 30%, at least about 40%, at least about 50%, or more compared to that achieved with the same effector target cell ratio with the same NK or T cells and target cells that are not brought into contact with the multispecific protein, as measured by an assay of NK or T cell activity, e.g., a marker of activation of NK cell cytotoxicity, CD107 or CD69 expression, IFNγ production, a classical in vitro chromium release test of cytotoxicity. Examples of protocols for activation and cytotoxicity assays are described in the Examples herein, as well as for example, in Pessino et al., J. Exp. Med, 1998, 188 (5): 953-960; Sivori et al., Eur J Immunol, 1999. 29:1656-1666; Brando et al., (2005) J. Leukoc. Biol. 78:359-371; EI-Sherbiny et al., (2007) Cancer Research 67(18):8444-9; and Nolte-'t Hoen et al., (2007) Blood 109:670-673).

In one aspect of the any of the embodiments herein, evaluating heterodimeric or heterotrimeric proteins for a characteristic of interest comprises evaluating the proteins for one or more properties selected from the group consisting of: binding to an antigen of interest, binding to an antigen on an immune effector cell, binding to a tumor, viral or bacterial antigen, binding to an FcRn receptor, binding to human CD16 and/or other Fc-domain mediated effector function(s), agonistic or antagonistic activity at a polypeptide to which the multimeric proteins binds, ability to modulate the activity (e.g. cause the death of) a cell expressing the antigen of interest, ability to direct a lymphocyte to a cell expressing the antigen of interest, ability to activate a lymphocyte in the presence and/or absence of a cell expressing the antigen of interest, NK cell activation, stability or half-life in vitro or in vivo, production yield, purity within a composition, and susceptibility to aggregate in solution.

In one aspect, the present disclosure provides a method for identifying or evaluating a protein, comprising the steps of:

(a) providing nucleic acid(s) encoding a protein described herein;

(b) expressing said nucleic acid(s) in a host cell to produce said protein, respectively; and recovering said protein; and

(c) evaluating the protein produced for a biological activity of interest, e.g., an activity disclosed herein, the ability to mediate the lysis of target cells (that express antigen of interest). In one embodiment, a plurality of different multispecific proteins are produced and evaluated.

In one embodiment, the step (c) comprises:

(i) testing the ability of the protein to cause effector cells (e.g. NK cells, T cells) that express an activating receptor bound by the protein to mediate the lysis of target cells, when incubated with such effector cells in the presence of target cells (that express antigen of interest). Optionally, step ( ) is followed by a step comprising: selecting a protein (e.g., for further development, for use as a medicament) that mediates the lysis of target cells.

In one aspect of any embodiment herein, a multispecific protein described herein that comprises and ABD that binds an activating receptor on an immune effector cell can for example be characterized by:

(a) being capable of inducing effector cells (e.g. T cell; NK cells) that express the activating receptor bound by the ABD of the multispecific protein to lyse target cells, when incubated in the presence of the effector cells and target cells; and

(b) lack of agonist activity at the activating receptor bound by the ABD when incubated with activating receptor-expressing effector cells in the absence of target cell; where the multispecific protein is capable of binding to CD16, the effector cells are CD16-negative cells, e.g. CD16-NK cells. Optionally, the effector cells are purified effector cells.

In one embodiment of any of the proteins disclosure herein, a multispecific heterotrimeric protein can be characterized as comprising a first polypeptide chain comprising an amino acid sequence which is at least 50%, 60%, 70%, 80%, 85%, 90%, 95%, 98 or 99% identical to the sequence of a first polypeptide chain of a F18, F19 or F20 protein disclosed herein (e.g. as sequence of SEQ ID NOS: 19, 22 or 25, respectively); a second polypeptide chain comprising an amino acid sequence which is at least 50%, 60%, 70%, 80%, 85%, 90%, 95%, 98 or 99% identical to the sequence of a second polypeptide chain of the respective F18, F19 or F20 protein disclosed herein (e.g. as sequence of SEQ ID NOS: 20, 23 or 26, respectively); and a third polypeptide chain comprising an amino acid sequence which is at least 50%, 60%, 70%, 80%, 85%, 90%, 95%, 98 or 99% identical to the sequence of a third polypeptide chain of a F18, F19 or F20 protein disclosed herein (e.g. as sequence of SEQ ID NOS: 18, 21 or 24, respectively). In one aspect, the protein comprises a dimeric Fc domain capable of being bound by a human CD16 polypeptide, e.g. a dimeric Fc domain comprising N-linked glycosylation at residue N297 (Kabat EU numbering). Optionally any or all of the variable regions or CDRs of the first, second and/or third chains are substituted with different variable regions. Optionally variable regions are excluded from the sequences that are considered for computing identity (e.g. the variable region may have any amino acid sequence); optionally wherein the anti-NKp46 variable regions or CDRs are included for computing identity and the variable regions or CDRs for the antigen binding domain that binds the other antigen (e.g. cancer antigens) are excluded from the sequences that are considered for computing identity.

### Uses of Compounds

In one aspect, provided is the use of any of the compounds defined herein, particularly the inventive multispecific proteins or antibodies and/or cells which express same for the manufacture of a pharmaceutical preparation for the treatment, prevention or diagnosis of a disease in a mammal in need thereof. Provided also are the use any of the compounds defined above as a medicament or an active component or active substance in a medicament. In a further aspect the invention provides methods for preparing a pharmaceutical composition containing a compound as defined herein, to provide a solid or a liquid formulation for administration orally, topically, or by injection. Such a method or process at least comprises the step of mixing the compound with a pharmaceutically acceptable carrier.

In one aspect, provided is a method to treat, prevent or more generally affect a predefined condition in an individual or to detect a certain condition by using or administering a multispecific protein described herein, or a (pharmaceutical) composition comprising same.

The polypeptides described herein can be used to prevent or treat disorders that can be treated with antibodies, such as cancers, solid and non-solid tumors, hematological malignancies, infections such as viral or microbial/bacterial infections, and inflammatory or autoimmune disorders.

In one embodiment, the protein of the disclosure binds one or more antigen of interest (e.g. two different antigens) expressed (e.g. co-expressed) on the surface of a malignant cell of a type cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors.

In one embodiment, the protein of the disclosure binds via two different antigen binding domains (e.g. via ABD₂ and ABD₃) to two different antigens co-expressed on the surface of a pathogenic cell (e.g. a tumor cell, a cancer-promoting cell, a cell in the tumor environment, tumor tissue or tumor-adjacent tissue), wherein at least one of the antigens is known to be expressed (e.g. at significant levels) on healthy cells or in healthy tissues. Preferably, if both antigen are known to be expressed (e.g. at significant levels) on healthy cells or in healthy tissues, they are not expressed by the same cells (co-expressed) at significant levels on healthy cells or in healthy tissues.

In one embodiment, the protein of the disclosure binds via two different antigen binding domains (e.g. via ABD₂ and ABD₃) to two different antigens co-expressed on the surface of a pro-inflammatory cell in an inflammatory disease.

In one embodiment, the inventive multispecific polypeptides described herein can be used to prevent or treat a cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary disorders that can be treated according to the invention include hematopoietic tumors of lymphoid lineage, for example T-cell and B-cell tumors.

In another aspect, the invention provides a method of restoring or potentiating the activity of immune effector cells (e.g. NK cells or T cells) in a patient in need thereof (e.g. a patient having a cancer, or a viral, parasite or bacterial infection), comprising the step of administering to the patient the multispecific protein. In one embodiment, this method is directed at increasing the activity of lymphocytes expressing the activating receptor bound by the multispecific protein.

In one aspect, the methods of treatment comprise administering to an individual a multispecific protein of the disclosure in a therapeutically effective amount. A therapeutically effective amount may be any amount that has a therapeutic effect in a patient having a disease or disorder (or promotes, enhances, and/or induces such an effect in at least a substantial proportion of patients with the disease or disorder and substantially similar characteristics as the patient).

The multispecific proteins of the disclosure can be included in kits. The kits may optionally further contain any number of polypeptides and/or other compounds, e.g., 1, 2, 3, 4, or any other number of multispecific proteins and/or other compounds. It will be appreciated that this description of the contents of the kits is not limiting in any way. For example, the kit may contain other types of therapeutic compounds. Optionally, the kits also include instructions for using the polypeptides, e.g., detailing the herein-described methods.

The invention also provides pharmaceutical compositions comprising the subject multispecific proteins and optionally other compounds as defined above. A multispecific protein and optionally another compound may be administered in purified form together with a pharmaceutical carrier as a pharmaceutical composition. The form depends on the intended mode of administration and therapeutic or diagnostic application. The pharmaceutical carrier can be any compatible, nontoxic substance suitable to deliver the compounds to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids A pharmaceutically acceptable carrier may further contain physiologically acceptable compounds that act for example to stabilize or to increase the absorption of the compounds Such physiologically acceptable compounds include, for example, carbohydrates, such as glucose, sucrose or dextrans, antioxidants, such as ascorbic acid or glutathione, chelating agents, low molecular weight proteins or other stabilizers or excipients One skilled in the art would know that the choice of a pharmaceutically acceptable carrier, including a physiologically acceptable compound, depends, for example, on the route of administration of the composition Pharmaceutically acceptable adjuvants, buffering agents, dispersing agents, and the like, may also be incorporated into the pharmaceutical compositions. Non-limiting examples of such adjuvants include by way of example inorganic and organic adjuvants such as alum, aluminum phosphate and aluminum hydroxide, squalene, liposomes, lipopolysaccharides, double stranded (ds) RNAs, single stranded(s-s) DNAs, and TLR agonists such as unmethylated CpG's.

Multispecific proteins according to the invention can be administered parenterally. Preparations of the compounds for parenteral administration must be sterile. Sterilization is readily accomplished by filtration through sterile filtration membranes, optionally prior to or following lyophilization and reconstitution. The parenteral route for administration of compounds is in accord with known methods, e.g. injection or infusion by intravenous, intraperitoneal, intramuscular, intraarterial, or intralesional routes. The compounds may be administered continuously by infusion or by bolus injection. A typical composition for intravenous infusion could be made up to contain 100 to 500 ml of sterile 0.9% NaCl or 5% glucose optionally supplemented with a 20% albumin solution and 1 mg to 10 g of the compound, depending on the particular type of compound and its required dosing regimen. Methods for preparing parenterally administrable compositions are well known in the art.

### EXAMPLES

### Example 1

### Generation of Anti-huNKp46 Antibodies

Balb/c mice were immunized with a recombinant human NKp46 extracellular domain recombinant-Fc protein. Mice received one primo-immunization with an emulsion of 50 μg NKp46 protein and Complete Freund Adjuvant, intraperitoneally, a 2nd immunization with an emulsion of 50 μg NKp46 protein and Incomplete Freund Adjuvant, intraperitoneally, and finally a boost with 10 μg NKp46 protein, intravenously. Immune spleen cells were fused 3 days after the boost with X63.Ag8.653 immortalized B cells, and cultured in the presence of irradiated spleen cells.

Primary screen: Supematant (SN) of growing clones were tested in a primary screen by flow cytometry using a cell line expressing the human NKp46 construct at the cell surface. Briefly, for FACS screening, the presence of reactive antibodies in supematants was revealed by Goat anti-mouse polyclonal antibody (pAb) labeled with PE.

A panel of antibodies that bound NKp46 was selected, produced and their variable regions sequenced and these antibodies and derivatives thereof further evaluated for their activity in the context of a bispecific molecule.

### Example 2

### Construction of Bispecific Antibody Formats

Different constructs were made for use in the preparation of bispecific antibodies. For anti-CD19 antigen binding domains, the DNA and amino acid sequences used were from the FDA-approved bispecific antibody blinatumomab (Blincyto™; AMG103; Amgen Corp.). For an antibody binding domain that binds NKp46, variable regions from antibodies specific for the NKp46 receptor reference NKp46-1 or NKp46-3 identified in Example 1 were used (also shown in Table 1). In a further example, an anti-CD20 antigen binding domain (i.e. for a second tumor antigen) used VH and VL sequences from the antibody GA101 (obinutuzumab; Gazyvaro®, Roche Pharmaceuticals). Proteins are cloned, produced and purified as described below. The anti-CD19 scFv and anti-CD20 scFv shown in the Table below are both shown in scFv configuration.

| | SEQ | |
|---|---|---|
| | ID | |
| | NO | Amino acid sequence |
| VH | 4 | QVQLQQSGPELVKPGASVKMSCKASGYTFTDYVINWGKQRSGQGLEWIG |
| anti-NKp46 | | EIYPGSGTNYYNEKFKAKATLTADKSSNIAYMQLSSLTSEDSAVYFCAR |
| (NKp46-1) | | RGRYGLYAMDYWGQGTSVTVSS |
| VL | 5 | DIQMTQTTSSLSASLGDRVTISCRASQDISNYLNWYQQKPDGTVKLLIY |
| anti-NKp46 | | YTSRLHSGVPSRFSGSGSGTDYSLTINNLEQEDIATYFCQQGNTRPWTF |
| (NKp46-1) | | GGGTKLEIK |
| VH | 8 | EVQLQQSGPELVKPGASVKISCKTSGYTFTEYTMHWVKQSHGKSLEWIG |
| anti-NKp46 | | GISPNIGGTSYNQKFKGKATLTVDKSSSTAYMELRSLTSEDSAVYYCAR |
| (NKp46-3) | | RGGSFDYWGQGTTLTVSS |
| VL | 9 | DIVMTQSPATLSVTPGDRVSLSCRASQSISDYLHWYQQKSHESPRLLIK |
| anti-NKp46 | | YASQSISGIPSRFSGSGSGSDFTLSINSVEPEDVGVYYCQNGHSFPLTF |
| (NKp46-3) | | GAGTKLELK |
| scFv | 16 | DIQLTQSPASLAVSLGQRATISCKASQSVDYDGDSYLNWYQQIPGQPPK |
| anti-CD19 | | LLIYDASNLVSGIPPRFSGSGSGTDFTLNIHPVEKVDAATYHCQQSTED |
| | | PWTFGGGTKLEIKGGGGSGGGGSGGGGSQVQLQQSGAELVRPGSSVKIS |
| | | CKASGYAFSSYWMNWVKQRPGQGLEWIGQIWPGDGDTNYNGKFKGKATL |
| | | TADESSSTAYMQLSSLASEDSAVYFCARRETTTVGRYYYAMDYWGQGTT |
| | | VTVSS |
| scFv | 17 | DIVMTQTPLSLPVTPGEPASISCRSSKSLLHSNGITYLYWYLQKPGQSP |
| anti-CD20 | | QLLIYQMSNLVSGVPDRFSGSGSGTDFTLKISRVEAEDVGVYYCAQNLE |
| | | LPYTFGGGTKVEIKGGGGSGGGGSGGGGSQVQLVQSGAEVKKPGSSVKV |
| | | SCKASGYAFSYSWINWVRQAPGQGLEWMGRIFPGDGDTDYNGKFKGRVT |
| | | ITADKSTSTAYMELSSLRSEDTAVYYCARNVFDGYWLVYWGQGTLVTVS |
| | | S |

### Cloning and Production of the Recombinant Proteins

Coding sequences are generated by direct synthesis and/or by PCR. PCR are performed using the PrimeSTAR MAX DNA polymerase (Takara) and PCR products are purified from 1% agarose gel using the NucleoSpin gel and PCR clean-up kit (Macherey-Nagel). Once purified the PCR product are quantified prior to the In-Fusion ligation reaction performed as described in the manufacturer's protocol (ClonTech). The plasmids are obtained after a miniprep preparation run on an EVO200 (Tecan) using the Nucleospin 96 plasmid kit (Macherey-Nagel). Plasmids are then sequenced for sequences confirmation before to transfecting the CHO cell line.

CHO cells are grown in the CD-CHO medium (Invitrogen) complemented with phenol red and 6 mM GlutaMax. The day before the transfection, cells are counted and seeded at 175.000 cells/ml. For the transfection, cells (200.000 cells/transfection) are prepared as described in the AMAXA SF cell line kit (AMAXA) and nucleofected using the DS137 protocol with the Nucleofector 4D device. All the transfections are performed using 300 ng of verified plasmids. After transfection, cells are seeded into 24 well plates in pre-warmed culture medium. After 24H, hygromycine B was added in the culture medium (200 μg/ml). Protein expression is monitored after one week in culture. Cells expressing the proteins are then sub-cloned to obtain the best producers. Sub-cloning is performed using 96 flat-bottom well plates in which the cells are seeded at one cell per well into 200 μl of culture medium complemented with 200 μg/ml of hygromycine B. Cells are left for three weeks before to test the clone's productivity.

Recombinant proteins which contain a IgG1-Fc fragment are purified using Protein-A beads (-rProteinA Sepharose fast flow, GE Healthcare). Briefly, cell culture supernatants are concentrated, clarified by centrifugation and injected onto Protein-A columns to capture the recombinant Fc containing proteins. Proteins are eluted at acidic pH (citric acid 0.1M pH3), immediately neutralized using TRIS-HCL pH8.5 and dialyzed against 1×PBS. Recombinant scFv which contain a “six his” tag are purified by affinity chromatography using Cobalt resin. Other recombinant scFv are purified by size exclusion chromatography (SEC).

### Format 18

Different constructs were made for use in the preparation of a bispecific protein using the variable domains DNA and amino acid sequences of the scFv specific for tumor antigen CD19 and the scFv specific for NKp46 identified described above. In addition, this protein binds to human CD16A (and other Fc gamma receptors) via its dimeric Fc domain. Proteins were cloned, produced and purified. Domains structures are shown in FIG. 1A.

Format 18 (F18): CD19-F18-NKp46-3

The domain structure of the trimeric F18 polypeptide is shown in FIG. 1A, wherein the interchain bonds between the CH1 and CK domains in the two Fc domain-containing chains are interchain disulfide bonds. The heterotrimer is made up of:

(1) a first (central) polypeptide chain having domains arranged as follows (N- to C-termini):

CH1-CH2-CH3-V_{H}^{anti-NKp46}-Cκ

and

(2) a second polypeptide chain having domains arranged as follows (N- to C-termini): Vκ ^{anti-NKp46}-CH1

and

(3) a third polypeptide chain having domains arranged as follows (N- to C-termini):

(V_{K}-V_{H})^{anti-CD19}-CK-CH2-CH3.

The resulting heterotrimer has a domain arrangement as follows, in which the Fc domain of the first and third polypeptide associate by CH3-CH3 dimerization and by CH1-Cκ dimerization, and wherein the first and second polypeptides associate by CH1-Cκ dimerization as well as VH-VK association:

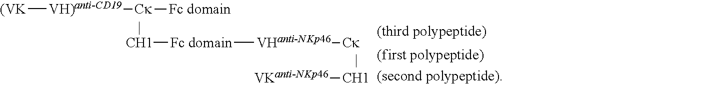

Proteins were cloned, produced and purified from cell culture supernatant by affinity chromatography using prot-A beads and analyzed and purified by SEC. The amino acid sequences of the three chains are shown below (anti-CD19 VH-VL pair underlined, anti-NKp46 VH-VL pair in bold and underined).

### CD19-F18-NKp46

Frag1 (third chain)
(SEQ ID NO: 18)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS
GGGSSRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGECDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYS
KLTVDKSRWQQGNVFSCSVMHEALHNHYTQKSLSLSPGK
Frag2 (first/central chain)
(SEQ ID NO: 19)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGSTGSEVQLQQSGPELVKPGAS
VKISCKTSGYTFTEYTMHWVKQSHGKSLEWIGGISPNIGGTSYNQKFKGK
ATLTVDKSSSTAYMELRSLTSEDSAVYYCARRGGSFDYWGQGTTLTVSSR
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC
Frag3 (second chain)
(SEQ ID NO: 20)
DIVMTQSPATLSVTPGDRVSLSCRASQSISDYLHWYQQKSHESPRLLIKY
ASQSISGIPSRFSGSGSGSDFTLSINSVEPEDVGVYYCQNGHSFPLTFGA
GTKLELKASTKGPSVFPLAPSSKSTSGGTAALGCLVKDYFPEPVTVSWNS
GALTSGVHTFPAVLQSSGLYSLSSVVTVPSSSLGTQTYICNVNHKPSNTK
VDKRVEPKSCDKTH

### Format 19

Different constructs were made for use in the preparation of a bispecific protein using the variable domains DNA and amino acid sequences of the scFv specific for tumor antigen CD19 and the scFv specific for NKp46 identified described above. In addition, this protein binds to human CD16A (and other Fc gamma receptors) via its dimeric Fc domain. Proteins were cloned, produced and purified. Domains structures are shown in FIG. 1B.

Format 19 (F19): CD19-F19-NKp46-3

The domain structure of the trimeric F19 polypeptide is shown in FIG. 1B, wherein the interchain bonds between the CH1 and Cκ domains in the two Fc domain-containing chains are interchain disulfide bonds. The heterotrimer is made up of:

(1) a first (central) polypeptide chain having domains arranged as follows (N- to C-termini):

(V_{K}—V_{H})^{anti-CD19}-CH1-CH2-CH3-V_{H}^{anti-NKp46}-Cκ

and

(2) a second polypeptide chain having domains arranged as follows (N- to C-termini):

Vκ ^{anti-NKp46}-CH1

and

(3) a third polypeptide chain having domains arranged as follows (N- to C-termini):

CK-CH2-CH3.

The resulting heterotrimer has a domain arrangement as follows, in which the Fc domain of the first and third polypeptide associate by CH3-CH3 dimerization and by CH1-Cκ dimerization, and wherein the first and second polypeptides associate by CH1-Cκ dimerization as well as VH-VK association:

Cκ-Fc domain (third polypeptide)

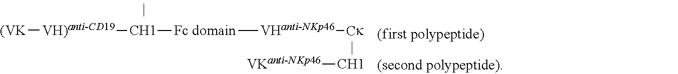

Proteins were cloned, produced and purified from cell culture supernatant by affinity chromatography using prot-A beads and analyzed and purified by SEC. The amino acid sequences of the three chains are shown below (anti-CD19 VH-VL pair underlined, anti-NKp46 VH-VL pair in bold and underlined).

### CD19-F9-NKp46

Frag1 (third polypeptide)
(SEQ ID NO: 21)
RTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGECDKTHTCPPCPAPELLGG
PSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGNVF
SCSVMHEALHNHYTQKSLSLSPGK
Frag2 (first/central polypeptide)
(SEQ ID NO: 22)
DIQLTQSPASLAVSLGQRATISCKASQSVDYDGDSYLNWYQQIPGQPPKLLIYDASNLVSGI

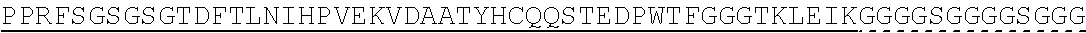

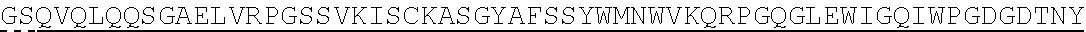

NGKFKGKATLTADESSSTAYMQLSSLASEDSAVYFCARRETTTVGRYYYAMDYWGQGTTVTV
SSGGGSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGSTGSEVQLQQSGPELVKPGASVKISCKTSGYTFTEY
TMHWVKQSHGKSLEWIGGISPNIGGTSYNQKFKGKATLTVDKSSSTAYMELRSLTSEDSAVY
YCARRGGSFDYWGQGTTLTVSSRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSF
NRGEC
Frag3 (second polypeptide)
(SEQ ID NO: 23)
DIVMTQSPATLSVTPGDRVSLSCRASQSISDYLHWYQQKSHESPRLLIKYASQSISGIPSRF
SGSGSGSDFTLSINSVEPEDVGVYYCQNGHSFPLTFGAGTKLELKASTKGPSVFPLAPSSKS
TSGGTAALGCLVKDYFPEPVTVSWNSGALTSGVHTFPAVLQSSGLYSLSSVVTVPSSSLGTQ
TYICNVNHKPSNTKVDKRVEPKSCDKTH

### Format 20

Another format, F20, was designed to test whether improved anti-tumor activity could be achieved through use of a tri-specific protein that binds NKp46, and targets two different antigens that are co-expressed by a tumor cell, via anti-cancer antigen ABDs that are placed in proximity within the multimeric protein (here, on the same termini in two associating polypeptide chains). The resulting protein is designed to have increased avidity for a tumor cell through binding to different antigens in settings where tumor cells present a limited number of available receptors. The protein can also or alternatively improve target cells specificity and decrease off-target toxicity (e.g. toward health cells) by binding to two different antigens that are co-expressed at the surface of the tumor cell but that are not co-expressed at the surface of healthy cells. The latter can be particularly advantageous when one or both of the antigens are known to be expressed on healthy cells.

Different constructs were made for use in the preparation of a trispecific antibody using for the variable domains DNA and amino acid sequences of the scFv specific for tumor antigen CD19, the VH and VL from antibody GA101 specific for tumor antigen CD20, and the scFv specific for NKp46 identified described above. In addition, this protein binds to human CD16A (and other Fc gamma receptors) via its dimeric Fc domain. Proteins were cloned, produced and purified. Domains structures are shown in FIG. 1C, wherein the interchain bonds between the CH1 and CK domains in the two Fc domain-containing chains are interchain disulfide bonds.

Format 20 (F20):

CD19-F20-NKp46-3

This protein incorporates two anti-CD19 ABD, each configured as an scFv and positioned at the end of one of the Fc domain-containing chains. The heterotrimer is made up of:

(1) a first (central) polypeptide chain having domains arranged as follows (N- to C-termini):

(VK-VH)^{anti-CD19}-CH1-CH2-CH3-V_{H}^{anti-NKp46}-Cκ

and

(2) a second polypeptide chain having domains arranged as follows (N- to C-termini): Vκ ^{anti-NKp46}-CH1

and

(3) a third polypeptide chain having domains arranged as follows (N- to C-termini):

(V_{K}-V_{H})^{anti-CD19}-Cκ-CH2-CH3.

The resulting heterotrimer has a domain arrangement as follows, in which the Fc domain of the first and third polypeptide associate by CH3-CH3 dimerization and by CH1-Cκ dimerization, and wherein the first and second polypeptides associate by CH1-Cκ dimerization as well as VH-VK association. The VH and VK pairs that form the N-terminal scFv in the first and second polypeptide were both ordered in the same orientation (as VK-VH, separated by a linker peptide) so as to avoid VH-VK association between the two anti-CD19 ABDs.

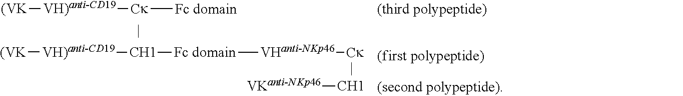

Proteins were cloned, produced and purified from cell culture supernatant by affinity chromatography using prot-A beads and analyzed and purified by SEC.

GA101-F20-NKp46-3

This protein incorporates two anti-CD20 ABD, each configured as an scFv and positioned at the end of one of the Fc domain-containing chains. The heterotrimer is made up of:

(1) a first (central) polypeptide chain having domains arranged as follows (N- to C-termini):

(VK-VH)^{anti-CD20}-CH1-CH2-CH3-V_{H}^{anti-NKp46}-Cκ

and

(2) a second polypeptide chain having domains arranged as follows (N- to C-termini): Vκ ^{anti-NKp46}-CH1

and

(3) a third polypeptide chain having domains arranged as follows (N- to C-termini):

(V_{K}-V_{H})^{anti-CD20}-Cκ-CH2-CH3.

The resulting heterotrimer has a domain arrangement as follows, in which the Fc domain of the first and third polypeptide associate by CH3-CH3 dimerization and by CH1-Cκ dimerization, and wherein the first and second polypeptides associate by CH1-Cκ dimerization as well as VH-VK association. The VH and VK pairs that form the N-terminal scFv in the first and second polypeptide were ordered VK-VH (separated by a linker peptide) so as to avoid VH-VK association between the two anti-CD20 ABDs.

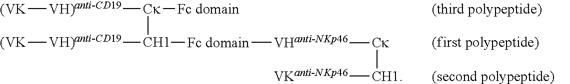

Proteins were cloned, produced and purified from cell culture supernatant by affinity chromatography using prot-A beads and analyzed and purified by SEC.

CD19-GA101-F20-NKp46-3

This protein incorporates one anti-CD19 ABD and one anti-CD20, each configured as an scFv and positioned at the end of one of the Fc domain-containing chains. The heterotrimer is made up of:

(1) a first (central) polypeptide chain having domains arranged as follows (N- to C-termini):

(VK-VH)^{anti-CD20}-CH1-CH2-CH3-V_{H}^{anti-NKp46}-Cκ

and

(2) a second polypeptide chain having domains arranged as follows (N- to C-termini): Vκ ^{anti-NKp46}-CH1

and

(3) a third polypeptide chain having domains arranged as follows (N- to C-termini):

(V_{K}-V_{H})^{anti-CD19}-Cκ-CH2-CH3.

The resulting heterotrimer has a domain arrangement as follows, in which the Fc domain of the first and third polypeptide associate by CH3-CH3 dimerization and by CH1-Cκ dimerization, and wherein the first and second polypeptides associate by CH1-Cκ dimerization as well as VH-VK association. The VH and VK pairs that form the N-terminal scFv in the first and second polypeptide were ordered VK-VH (separated by a linker peptide) so as to avoid VH-VK association between the anti-CD19 ABD and the anti-CD20 ABD.

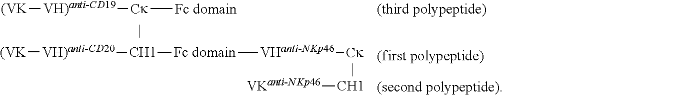

Proteins were cloned, produced and purified from cell culture supernatant by affinity chromatography using prot-A beads and analyzed and purified by SEC.

The amino acid sequences of the three chains are shown below (anti-CD9 VH-VL pair underlined, anti-NKp46 VH-VL pair in bold and underlined, and the anti-CD20VH-VL pair in italics and underlined (dotted)).

CD19-CD20-r20-NKp46 (also referrdtoas CD9-GA11-20-NKp46)

Frag1 (third polypeptide)
(SEQ ID NO: 24)
DIQLTQSPASLAVSLGQRATISCKASQSVDYDGDSYLNWYQQIPGQPPKLLIYDASNLVSGI

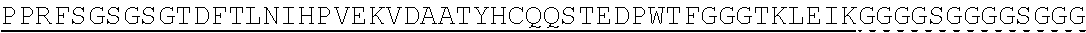

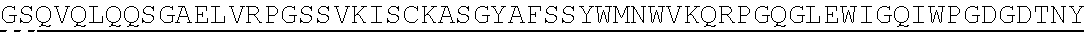

NGKFKGKATLTADESSSTAYMQLSSLASEDSAVYFCARRETTTVGRYYYAMDYWGQGTTVTV
SSGGGSSRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGECDKTHTCPPCP
APELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Frag2 (first/central polypeptide)
(SEQ ID NO: 25)

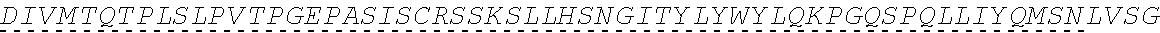

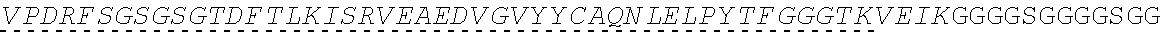

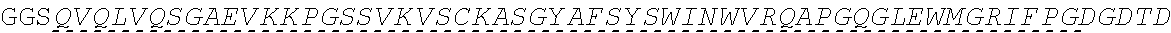

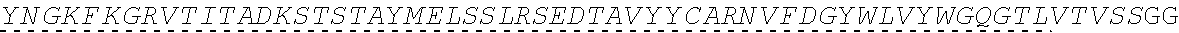

GSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPELLGGP
SVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFS
CSVMHEALHNHYTQKSLSLSPGSTGSEVQLQQSGPELVKPGASVKISCKTSGYTFTEYTMHW
VKQSHGKSLEWIGGISPNIGGTSYNQKFKGKATLTVDKSSSTAYMELRSLTSEDSAVYYCAR
RGGSFDYWGQGTTLTVSSRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGE
C
Frag3 (second polypeptide)
(SEQ ID NO: 26)
DIVMTQSPATLSVTPGDRVSLSCRASQSISDYLHWYQQKSHESPRLLIKYASQSISGIPSRF
SGSGSGSDFTLSINSVEPEDVGVYYCQNGHSFPLTFGAGTKLELKASTKGPSVFPLAPSSKS
TSGGTAALGCLVKDYFPEPVTVSWNSGALTSGVHTFPAVLQSSGLYSLSSVVTVPSSSLGTQ
TYICNVNHKPSNTKVDKRVEPKSCDKTH

### Example 3

### Tumor Target Celllysis

NKp46 xCD19 bispecific proteins having an arrangement according to the F18, F19 or F20 formats described in Example 2 with anti-NKp46 variable domains from NKp46-1 were for their functional ability to direct fresh human purified NK cells to lyse Daudi tumor target cells (co-expressing CD19 and CD20).

Briefly, the cytolytic activity of fresh human purified NK cells from EFS Buffy Coat was assessed in a classical 4-h ⁵¹Cr-release assay in U-bottom 96 well plates. Daudi cells were labelled with ⁵¹Cr (50 μCi (1.85 MBq)/1×10⁶ cells), then mixed with NK cells at an effector/target ratio equal to 50:1, in the presence of test antibodies at dilution ranges starting from 10⁻⁷ mol/L with 1/5 dilution (n=8 concentrations). were labelled with ⁵¹Cr and then mixed with NK cells at an effector/target ratio equal to 10:1, in the presence of test antibodies at dilution range starting from 10 μg/ml with 1/10 dilution (n=8 concentrations).

After brief centrifugation and 4 hours of incubation at 37° C., 50 μL of supernatant were removed and transferred into a LumaPlate (Perkin Elmer Life Sciences, Boston, Mass.), and ⁵¹Cr release was measured with a TopCount NXT beta detector (PerkinElmer Life Sciences, Boston, Mass.). All experimental conditions were analyzed in triplicate, and the percentage of specific lysis was determined as follows: 100×(mean cpm experimental release−mean cpm spontaneous release)/(mean cpm total release−mean cpm spontaneous release). Percentage of total release is obtained by lysis of target cells with 2% Triton X100 (Sigma) and spontaneous release corresponds to target cells in medium (without effectors or Abs).

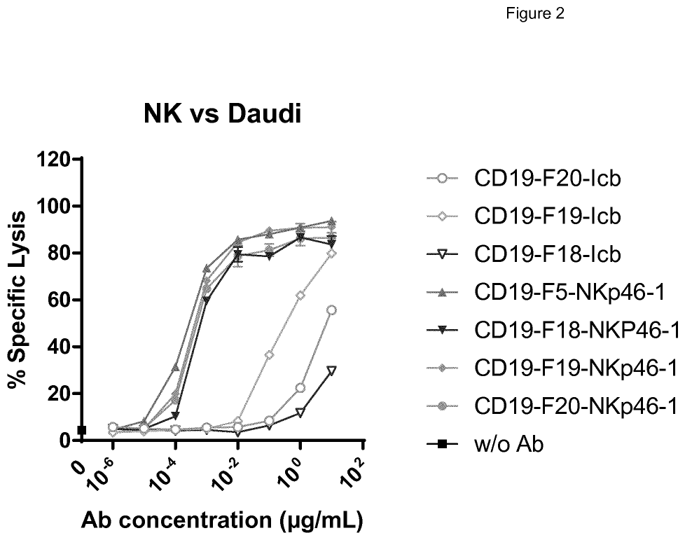

The results of these experiments are shown in FIG. 2. Each of proteins F18, F19 and F20 formats were highly effective in mediating target cell lysis by NK cells, with an EC₅₀ of about 1 picomolar.

FIG. 2 shows target cell lysis mediated by the CD19-F18-NKp46 (bispecific protein in F18 format) whose Fc domain binds CD16. The F18 format was highly effective in mediating target cell lysis by NK cells, and was more potent in mediating Daudi target cell lysis than a variant of the F18 format bispecific protein designated “CD19-F18-Icb” in which the NKp46 VH/VL pair were replaced by a VH/VL pair whose target is absent. “Icb” refers to the beziotoxumab-derived antigen binding domain which is non-functional in this experimental setting due to absence of its target antigen (antibody bezlotoxumab is specific for the Clostridum difficile toxin B; trade name Zinplava™).

FIG. 2 further shows target cell lysis mediated by the CD9-F19-NKp46 (bispecific protein in F19 format) whose Fc domain binds CD16. The F19 format was highly effective in mediating target cell lysis by NK cells, and was more potent in mediating Daudi target cell lysis than a variant of the F19 format bispecific protein designated “CD19-F19-Icb” in which the NKp46 VH/VL pair were replaced by a VH/VL pair using the bezlotoxumab-derived antigen binding domain whose target is absent in this setting.

FIG. 2 further shows target cell lysis mediated by the CD19-CD29-F20-NKp46 (trispecific protein in F20 format) whose Fc domain binds CD16. The F20 format was highly effective in mediating target cell lysis by NK cells, and was more potent in mediating Daudi target cell lysis than a variant of the F20 format bispecific protein designated “CD19-CD20-F20-Icb” in which the NKp46 VH/VL pair were replaced by a VH/VL pair using the bezlotoxumab-derived antigen binding domain whose target is absent in this setting.

### Example 4

### Improved Anti-Tumor Activity by Targeting Different Co-Expressed Antigens

Different NKp46×CD20 using the identical anti-NKp46 variable domains from antibody NKp46-1 were compared for their functional ability to direct human purified NK cells to lyse Daudi tumor target cells (co-expressing CD19 and CD20). Among the comparators, we included F5 proteins as described in PCT publication no. WO2016/207278, particularly potent NK cell engagers, as well as full-length human IgG1 antibodies capable of bivalent binding to their CD19 target (antibody having the VH/VL of rituximab) or CD20 target (antibody having the VH/VL of obinutuzumab) via the same VH and VL sequences as the bispecific proteins. The bispecific proteins tested included proteins that possessed one or two antigen binding domains specific for CD20, with or without ability to bind human CD16, and proteins possessing both an anti-CD19 and an anti-CD20 ABD, placed either in cis or trans with respect to the Fc domain, including proteins in which the CD19 and CD20 domains were inverted when in the trans configuration. Cytolytic activity of fresh human purified NK cells from EFS Buffy Coat was assessed in a classical 4-h ⁵¹Cr-release assay as in Example 3.

The test proteins included:

CD20-F5-NKp46-1: proteins of “F5” configuration described in WO2016/207278 having one anti-CD20 ABD derived from GA101 and one anti-NKp46 ABD, separated by a dimeric Fc domain that binds human CD16.

GA101-T5-NKp46-1-CD19: protein of “T5” configuration described in WO2016/207278 having one anti-CD20 ABD derived from GA101, a dimeric Fc domain that binds human CD16, one anti-NKp46 ABD, and one anti-CD19 ABD, wherein the Fc domain is interposed between the CD20 and CD19 ABD (the CD20 and CD19 ABD are placed in trans with respect to the Fc domain).

CD20-F18-NKp46-1: protein of “F18” configuration described in Example 2, having one anti-CD20 ABD derived from GA101 and one anti-NKp46 ABD from antibody NKp46-1, separated by a dimeric Fc domain that binds human CD16.

CD20-F19-NKp46-1: protein of “F19” configuration described in Example 2, having one anti-CD20 ABDs and one anti-NKp46 ABD from antibody NKp46-1, separated by a dimeric Fc domain that binds human CD16.

CD20-F20-NKp46-1: protein of “F20” configuration described in Example 2, having two anti-CD20 ABD derived from GA101 and one anti-NKp46 ABD from antibody NKp46-1, separated by a dimeric Fc domain that binds human CD16.

CD19-F20-NKp46-1: protein of “F20” configuration described in Example 2, having two anti-CD19 ABDs and one anti-NKp46 ABD from antibody NKp46-1, separated by a dimeric Fc domain that binds human CD16.

CD19-CD20-F20-NKp46-1: protein of “F20” configuration described in Example 2, having one anti-CD20 ABD derived from GA101 and one anti-CD19 ABD placed in cis with respect to the Fc domain, a dimeric Fc domain that binds human CD16, and one anti-NKp46 ABD from antibody NKp46-1, wherein the Fc domain is interposed between the C-terminal NKp46 ABD the N-terminal CD19 and CD20 ABDs.

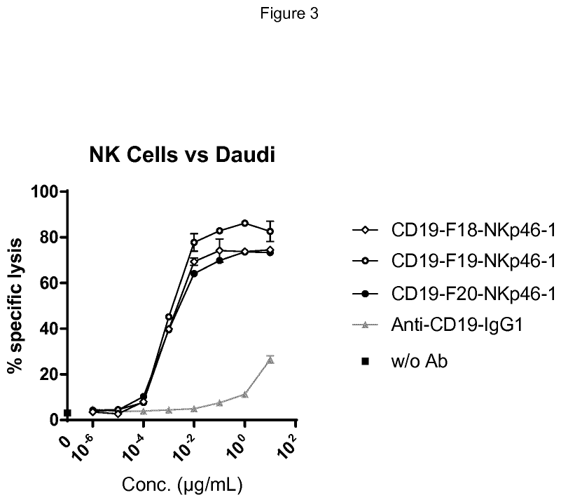

Results are shown in FIGS. 3 to 7, showing the % specific lysis of tumor cells induced by the test proteins as a function of protein concentration.

FIG. 3 shows a comparison of CD19-F18-NKp46-1, CD19-F19-NKp46-1 and CD19-F20-NKp46-1, and full-length anti-CD19 antibodies which engage CD16 (but not NKp46). Each of the F18, F19 and F20 proteins were highly potent in inducing NK cell mediated lysis of tumor target cells, and all were more potent than the full-length anti-CD19 antibody. The monovalent binding proteins F18 and F19 that bind to CD19 via one ABD were as efficient in mediating target cells lysis as the bivalent binding F20 protein.

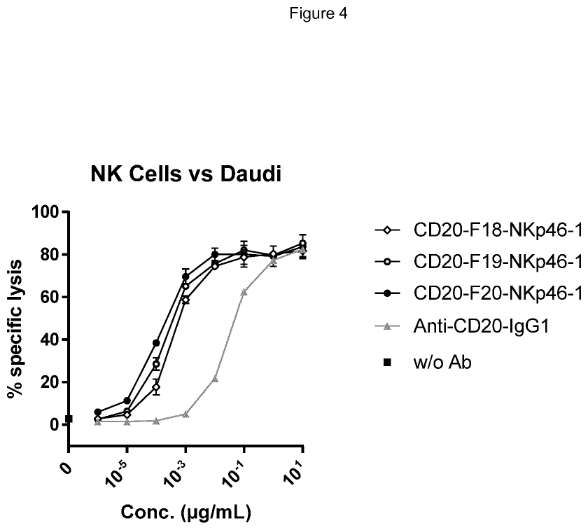

FIG. 4 shows a comparison of CD20-F18-NKp46-1, CD20-F19-NKp46-1 and CD20-F20-NKp46-1, and full-length anti-CD20 antibodies which engage CD16 (but not NKp46). Each of the F18, F19 and F20 proteins were highly potent in inducing NK cell mediated lysis of tumor target cells, and all were more potent than the full-length anti-CD20 antibody. Once again, the monovalent binding proteins F18 and F19 that bind to CD20 via one ABD were as efficient in mediating target cells lysis as the bivalent binding F20 protein.

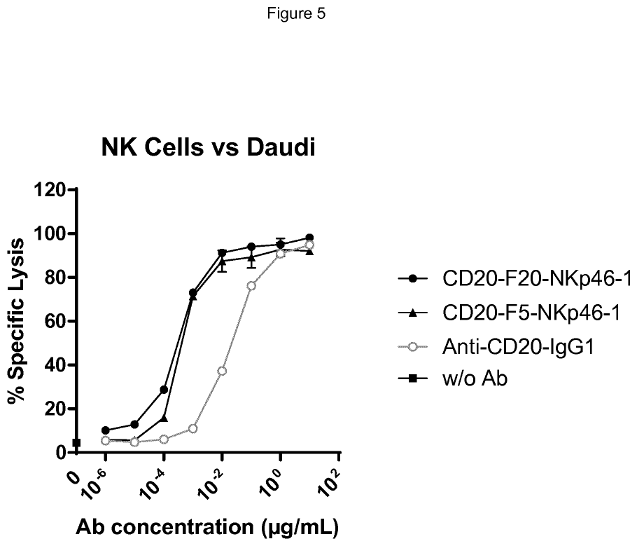

FIG. 5 shows a comparison of the bivalent binding CD20-F20-NKp46-1 protein and the highly potent monovalent binding CD20-F5-NKp46-1 protein, as well as full-length anti-CD20 antibodies which engage CD16 (but not NKp46). F5 and F20 proteins were similarly potent in inducing NK cell mediated lysis of tumor target cells, and both were more potent than the full-length anti-CD20 antibody.

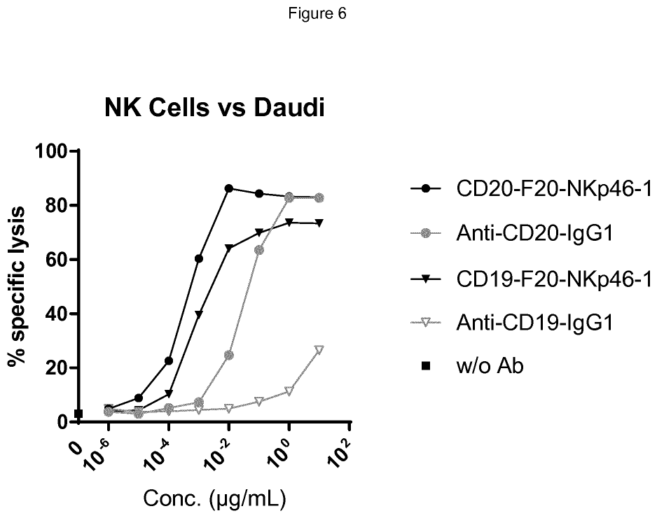

FIG. 6 shows a comparison of bivalent binding F20 proteins specific for either CD19 or CD20. Both F20 proteins are far more potent than bivalent binding full-length anti-CD19 or CD20 IgG1 antibodies, and the CD20-F20-NKp46-1 is somewhat more potent than CD19-F20-NKp46-1 protein in inducing target cell lysis of Daudi tumor cells. The full-length anti-CD20 antibodies were more potent that the full-length anti-CD19 IgG1 antibodies.

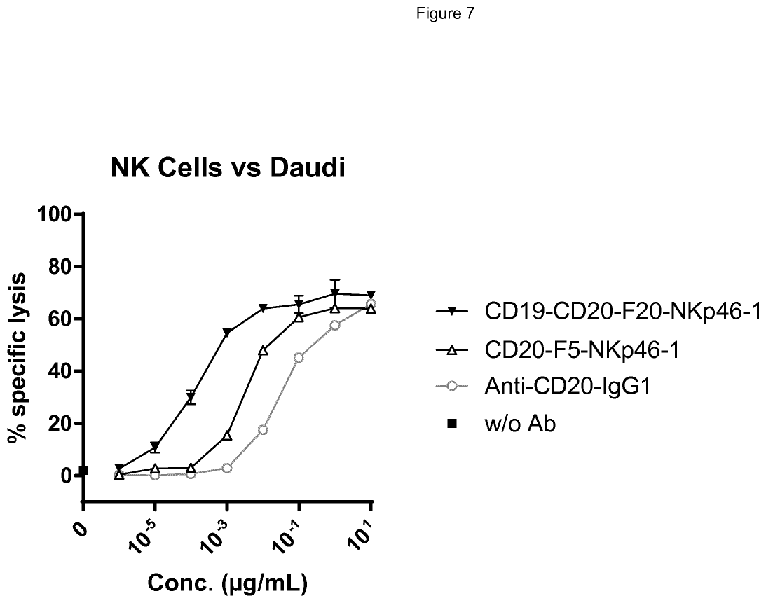

FIG. 7 shows a comparison of the bivalent binding CD20-F20-NKp46-1 protein and the most active protein of FIG. 6 (the F5 protein that included one anti-CD20 ABD, referred to as CD19-CD20-F20-NKp46-1 protein). Full-length anti-CD20 antibodies which engage CD16 (but not NKp46) were included as comparator. Surprisingly, despite the anti-CD19 full-length antibodies having lower activity (see FIG. 6) and the CD19-F20-NKp46-1 protein having lower potency that its anti-CD20 equivalent, the CD19-CD20-F20-NKp46-1 protein that binds monovalently to CD19 and monovalently to CD20 showed a dramatic increase in potency (between 1 and 2-log increase in potency based on EC₅₀ values) in mediating Daudi tumor cell lysis than the CD20-F5-NKp46-1 protein.

Engaging different antigens monovalently can therefore provide an improvement in potency even among the most potent bispecific proteins; furthermore considerable additional potency is conferred by binding the two different antigens via ABDs positioned in greater proximity, here positioned at a common terminus with respect to the Fc domain (both CD19 and CD20 are positioned N-terminal to the Fc domain whereas the anti-NKp46 ABD is positioned C-terminal to the Fc domain).

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e. g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as reference to an element or elements is intended to provide support for a similar aspect or embodiment of the invention that “consists of,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-45. (canceled)

46. A multispecific protein that binds a first, second and third antigen of interest, comprising a first, second and third polypeptide chain, comprising:
i) a multispecific protein comprising a first (central) chain comprising, from N- to C-terminus, an antigen binding domain that binds the second antigen of interest, a first CH1 or Cκ domain, an Fc region, and a variable domain fused to a second CH1 or Cκ domain (forming a V-(CH1/Cκ) unit);
a second chain comprising, from N- to C-terminus, a variable domain fused to a CH1 or Cκ domain, wherein the variable domain and CH1 or Cκ domain are complementary to the respective variable domain and second CH1 or Cκ domain of the first chain (the V-(CH1/Cκ) unit), such that the second chain binds to the first chain by CH1-Cκ dimerization and VH-VK association, wherein the VH and VK together form a first antigen binding domain that binds a the first antigen of interest; and
a third chain comprising, from N- to C-terminus, an antigen binding domain that binds the third antigen of interest, a CH1 or Cκ domain and a Fc region, wherein said CH1 or Cκ domain is complementary to the first CH1 or Cκ domain of the first chain, such that the third chain binds to the first chain by CH1-Cκ dimerization and CH3-CH3 dimerization, wherein the first antigen of interest is an activating receptor expressed at the surface of an immune cell, wherein the second antigen of interest is a first cancer antigen, and wherein the third antigen of interest is a second cancer antigen, wherein the first and second cancer antigen are different from one another, optionally wherein the first and second cancer antigen are co-expressed by a malignant cell; or
ii) a heterotrimeric multispecific protein comprising a dimeric Fc domain, having the domain arrangement: 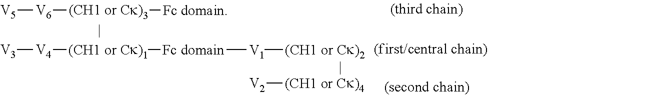
wherein one of (CH1 or Cκ)₁ and (CH1 or Cκ)₃ is a CH1 domain and the other is a Cκ domain, wherein one of (CH1 or Cκ)₂ and (CH1 or Cκ)₄ is a CH1 domain and the other is a Cκ domain, wherein one V₁ and V₂ is a light chain variable domain and the other is a heavy chain variable domain, wherein the V₁ and V₂ pair associate with one another to form an ABD₁ that binds a first antigen of interest, wherein one V₃ and V₄ is a light chain variable domain and the other is a heavy chain variable domain, wherein the V₃ and V₄ pair associate with one another to form an ABD₂ that binds a second antigen of interest, and wherein one V₅ and V₆ is a light chain variable domain and the other is a heavy chain variable domain, wherein the V₅ and V₆ pair associate with one another to form an ABD₃ that binds a third antigen of interest; or
iii) a multispecific protein comprising a first, second and third polypeptide chain, comprising:
a first (central) chain comprising a first CH1 or Cκ domain fused to an Fc region, and a variable domain fused to a second CH1 or Cκ domain (forming a V-(CH1/Cκ) unit);
a second chain comprising a variable domain fused to a CH1 or Cκ domain, wherein the variable domain and CH1 or Cκ domain are complementary to the respective variable domain and CH1 or Cκ domain of the first chain, such that the second chain binds to the first chain by CH1-Cκ dimerization and VH-VK association, wherein the VH and VK together form a first antigen binding domain that binds a first antigen of interest; and
a third chain comprising a CH1 or Cκ domain fused to an Fc region, wherein said CH1 or Cκ domain is complementary to the first CH1 or Cκ domain of the first chain, such that the third chain binds to the first chain by CH1-Cκ dimerization and CH3-CH3 dimerization; or
iv) a multispecific antigen binding protein comprising:
(a) a first antigen binding domain (ABD) that binds to a human NK cell activating receptor NKp46, NKp30 or NKG2D,
(b) a second ABD that binds to a first pre-determined antigen of interest,
(c) a third ABD that binds to a second, different, pre-determined antigen of interest, where the second and third ABD each bind to a different target antigen co-expressed at the surface of a tumor cell to be eliminated, optionally wherein one or both of the target antigens are known to also be expressed by healthy cells,
(d) and a CD16A binding polypeptide, optionally an Fc polypeptide or portion thereof capable of binding human CD16A,
wherein the CD16A binding polypeptide is interposed between the first ABD and the second and third ABDs, optionally wherein the first ABD is positioned C-terminal to the CD16A binding polypeptide and the second and third ABD are both positioned N-terminal to the CD16A binding polypeptide, wherein the multispecific protein is capable of directing an NK cell expressing said activating receptor to lyse a target cell co-expressing the first and second antigen of interest, wherein said lysis of the target cell is mediated by said activating receptor signaling.

47. The multispecific protein of claim 46, wherein said multispecific protein is a heterotrimeric multispecific protein comprising a dimeric Fc domain, having the domain arrangement: 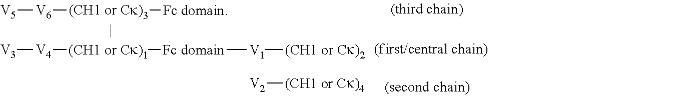
wherein one of (CH1 or Cκ)₁ and (CH1 or Cκ)₃ is a CH1 domain and the other is a Cκ domain, wherein one of (CH1 or Cκ)₂ and (CH1 or Cκ)₄ is a CH1 domain and the other is a Cκ domain, wherein one V₁ and V₂ is a light chain variable domain and the other is a heavy chain variable domain, wherein the V₁ and V₂ pair associate with one another to form an ABD₁ that binds a first antigen of interest, wherein one V₃ and V₄ is a light chain variable domain and the other is a heavy chain variable domain, wherein the V₃ and V₄ pair associate with one another to form an ABD₂ that binds a second antigen of interest, and wherein one V₅ and V₆ is a light chain variable domain and the other is a heavy chain variable domain, wherein the V₅ and V₆ pair associate with one another to form an ABD₃ that binds a third antigen of interest.

48. The multispecific protein of claim 46, wherein said multispecific protein comprises a first, second and third polypeptide chain, comprising:
a first (central) chain comprising a first CH1 or Cκ domain fused to an Fc region, and a variable domain fused to a second CH1 or Cκ domain (forming a V-(CH1/Cκ) unit);
a second chain comprising a variable domain fused to a CH1 or Cκ domain, wherein the variable domain and CH1 or Cκ domain are complementary to the respective variable domain and CH1 or Cκ domain of the first chain, such that the second chain binds to the first chain by CH1-Cκ dimerization and VH-VK association, wherein the VH and VK together form a first antigen binding domain that binds a first antigen of interest; and
a third chain comprising a CH1 or Cκ domain fused to an Fc region, wherein said CH1 or Cκ domain is complementary to the first CH1 or Cκ domain of the first chain, such that the third chain binds to the first chain by CH-Cκ dimerization and CH3-CH3 dimerization.

49. The protein of claim 48, wherein the first antigen of interest is an activating receptor expressed at the surface of an effector cell.

50. The protein of claim 49, wherein the activating receptor is NKp46.

51. The protein of claim 49, wherein the second and/or third antigen of interest a cancer, viral or bacterial antigen.

52. The multispecific protein of claim 46, wherein said multispecific protein is a multispecific antigen binding protein comprising:
(a) a first ABD that binds to a human NK cell activating receptor NKp46, NKp30 or NKG2D,
(b) a second ABD that binds to a first pre-determined antigen of interest,
(c) a third ABD that binds to a second, different, pre-determined antigen of interest, where the second and third ABD each bind to a different target antigen co-expressed at the surface of a tumor cell to be eliminated, optionally wherein one or both of the target antigens are known to also be expressed by healthy cells,
(d) and a CD16A binding polypeptide, optionally an Fc polypeptide or portion thereof capable of binding human CD16A,
wherein the CD16A binding polypeptide is interposed between the first ABD and the second and third ABDs, optionally wherein the first ABD is positioned C-terminal to the CD16A binding polypeptide and the second and third ABD are both positioned N-terminal to the CD16A binding polypeptide, wherein the multispecific protein is capable of directing an NK cell expressing said activating receptor to lyse a target cell co-expressing the first and second antigen of interest, wherein said lysis of the target cell is mediated by said activating receptor signaling.

53. A nucleic acid or set of nucleic acids encoding a multispecific protein according to claim 46.

54. A recombinant host cell expressing a protein or nucleic acid(s) of claim 53.

55. A pharmaceutical composition comprising a protein according to claim 46 and a pharmaceutically acceptable carrier.

56. A method of treating a disease in a subject comprising administering to the subject a composition of claim 46.

57. The method of claim 56, wherein the disease is a cancer.

58. A method of making a multimeric protein comprising expressing said multimeric protein in a host cell of claim 54 to produce said multimeric protein; loading the protein produced onto an affinity purification support, optionally a Protein-A support, and recovering said multimeric protein.

59. A method for identifying or evaluating a multimeric polypeptide, comprising the steps of expressing said multimeric protein in a host cell of claim 54, recovering said multimeric protein and evaluating the polypeptide produced for a biological activity of interest.


SEQUENCE LISTING (30 sequences)

SEQ ID NO: 1 - protein, 304 aa, homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 2 - protein, 201 aa, homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 3 - protein, 216 aa, homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 4 - protein, 120 aa, mus musculus
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 5 - protein, 107 aa, mus musculus
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 6 - protein, 122 aa, mus musculus
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 7 - protein, 107 aa, mus musculus
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 8 - protein, 116 aa, mus musculus
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 9 - protein, 107 aa, mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 10 - protein, 117 aa, mus musculus
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 11 - protein, 107 aa, mus musculus
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 12 - protein, 115 aa, mus musculus
QVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSS

SEQ ID NO: 13 - protein, 107 aa, mus musculus
DIQMTQSPSSLSASLGERVSLTCRASQDIGSSLNWLQQEPDGTIKRLIYATSSLDSGVPKRFSGSRSGSDYSLTISSLESEDFVDYYCLQYASSPWTFGGGTKLEIK

SEQ ID NO: 14 - protein, 120 aa, mus musculus
DVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSS

SEQ ID NO: 15 - protein, 107 aa, mus musculus
DIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 16 - protein, 250 aa, Artificial
  Chimeric
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 17 - protein, 246 aa, Artificial
  Chimeric
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKGGGGSGGGGSGGGGSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 18 - protein, 589 aa, Artificial
  Homo-sapiens derived
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 556 aa, Artificial
  Homo-sapiens derived
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 214 aa, Artificial
  Homo-sapiens derived
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 21 - protein, 333 aa, Artificial
  Homo-sapiens derived
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 811 aa, Artificial
  Homo-sapiens derived
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 214 aa, Artificial; identical to SEQ ID NO: 20
  Homo-sapiens derived

SEQ ID NO: 24 - protein, 589 aa, Artificial; identical to SEQ ID NO: 18
  Homo sapiens derived

SEQ ID NO: 25 - protein, 807 aa, Artificial
  Homo sapiens derived
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKGGGGSGGGGSGGGGSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSGGGSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, Artificial; identical to SEQ ID NO: 20
  Homo sapiens derived

SEQ ID NO: 27 - protein, 13 aa, homo sapiens
THTCPPCPAPELL

SEQ ID NO: 28 - protein, 13 aa, homo sapiens
THTCSSCPAPELL

SEQ ID NO: 29 - protein, 15 aa, homo sapiens
NASSMCVPSPAPELL

SEQ ID NO: 30 - protein, 13 aa, homp sapiens
THTSPPSPAPELL